(19) United States
(12) Patent Application Publication
(10) Pub. No.: US 2023/0392160 A1
(43) Pub. Date: Dec. 7, 2023

(54) COMPOSITIONS AND METHODS FOR INCREASING GENOME EDITING EFFICIENCY

(71) Applicant: John Innes Centre, Norwich (GB)

(72) Inventor: Tom Lawrenson, Norwich (GB)

(21) Appl. No.: 18/298,234

(22) Filed: Apr. 10, 2023

Related U.S. Application Data
(60) Provisional application No. 63/330,106, filed on Apr. 12, 2022.
(60) Provisional application No. 63/386,452, filed on Dec. 7, 2022.

(51) Int. Cl. C12N 15/82; C12N 15/11; C12N 9/22; C12Q 1/6895

(52) U.S. Cl. CPC C12N 15/8273; C12N 15/11; C12N 9/22; C12Q 1/6895; C12N 15/8213; C12N 2310/20; C12N 2800/80; C12Q 2600/13; C12Q 1/6869

(57) ABSTRACT

Provided are compositions and methods for improving gene editing efficiency in plants. Methods and compositions are also provided for producing modifications using novel Cas12a nuclease variants. Modified plant cells and plants comprising DNA and protein compositions of novel Cas12a nuclease variants are further provided.

### CROSS REFERENCE TO RELATED APPLICATIONS

This application claims the benefit of U.S. Provisional Application No. 63/330,106, filed on Apr. 12, 2022, and U.S. Provisional Application No. 63/386,452, filed on Dec. 7, 2022, the entire content of each of which is hereby incorporated herein by reference.

### INCORPORATION OF SEQUENCE LISTING

A sequence listing containing the file named “AGOE008US_ST26.xml” which is 94 kilobytes (measured in MS-Windows®) and created on Apr. 6, 2023, and comprises 58 sequences, is incorporated herein by reference in its entirety.

### FIELD OF THE INVENTION

The present disclosure relates to the field of plant molecular biology and plant genetic engineering, and to methods and compositions for genome editing in plants. In particular, the invention relates to novel Cas12a nuclease variants and methods of improving gene editing efficiency. Plant genetic engineering methods are used to modify Cas12a DNA and the encoded proteins, and to transfer these molecules into plants of agronomic importance. More specifically, the invention comprises DNA and protein compositions of novel LbCas12a nuclease variants, and to the plants containing these compositions.

### BACKGROUND OF THE INVENTION

Precise genome editing technologies are powerful tools for engineering gene expression and modulating protein function and have the potential to improve important agricultural traits. In particular, the clustered regularly interspaced short palindromic repeats (CRISPR)-Cas9 system has revolutionized the field of genome editing. However, the editing efficiency of this powerful tool is still very low in some plant species. Therefore, a continuing need exists in the art to develop novel compositions and methods to increase the efficiency of genome editing in plants.

### SUMMARY

In one aspect, the present disclosure provides recombinant DNA molecules comprising a polynucleotide sequence selected from the group consisting of: (a) a sequence with at least 85 percent identity to any of SEQ ID NOs:1, 3, 5, 7, and 8; (b) a sequence comprising SEQ ID NOs:1, 3, 5, 7, and 8; (c) a fragment of any of SEQ ID NOs:1, 3, 5, 7, and 8; and (d) a sequence encoding a protein having at least 85 percent identity to any of SEQ ID NOs: 2, 4, 6, and 9. In some embodiments, the protein encoded by said polynucleotide sequence comprises a modification at amino acid position 156 as compared to a protein comprising the amino acid sequence of SEQ ID NO:46. For example, recombinant DNA molecules having at least 90 percent identity or at least 95 percent identity to any of SEQ ID NOs:1, 3, 5, 7, and 8 and encoding a protein having a modification at amino acid position 156 as compared to a protein comprising the amino acid sequence of SEQ ID NO:46. In some embodiments, recombinant DNA molecules provided herein comprise any of SEQ ID NOs:1, 3, 5, 7, and 8. In certain examples, the modification at amino acid position 156 relative to SEQ ID NO: 46 is further defined as an aspartate to arginine substitution.

In another aspect, the present disclosure provides recombinant DNA molecules comprising a polynucleotide sequence selected from the group consisting of: a) a sequence with at least 85 percent identity to any of SEQ ID NOs:1, 3, 5, 7, and 8; b) a sequence comprising SEQ ID NOs:1, 3, 5, 7, and 8; c) a fragment of any of SEQ ID NOs:1, 3, 5, 7, and 8; and d) a sequence encoding a protein having at least 85 percent identity to any of SEQ ID NOs: 2, 4, 6, and 9, and further comprising at least one intron sequence having a sequence of any of SEQ ID NOs:10-17. In some embodiments, polynucleotides provided herein comprise one or more intron sequences of any of SEQ ID NOs:10-17.

In yet another aspect, transgenic plant cells comprising the recombinant DNA molecules provided herein are described. Transgenic plant cells provided may be monocotyledonous plant cells, including but not limited to barley, B. oleracea, wheat, and corn cells. Transgenic plant cells provided may also be dicotyledonous plant cells. Further provided are transgenic plants, or parts thereof, comprising the recombinant DNA molecule described herein. Progeny plants comprising the DNA molecules provided herein are further described. The instant disclosure further provides transgenic seeds comprising the recombinant DNA molecules described herein.

The recombinant DNA molecules described herein may be expressed in a plant cell to produce a genomic modification and may also be in operable linkage with a vector, wherein said vector is selected from the group consisting of a plasmid, phagemid, bacmid, cosmid, and a bacterial or yeast artificial chromosome.

Recombinant DNA molecules provided herein may be present within a host cell, wherein said host cell is any type of cell. Host cells contemplated by the present disclosure include cells selected from the group consisting of a bacterial cell, an animal cell, a plant cell, a yeast cell, a fungal cell, and an insect cell. For example, the bacterial host cell may be from a genus of bacteria selected from the group consisting of Agrobacterium, Rhizobium, Bacillus, Brevibacillus, Escherichia, Pseudomonas, Klebsiella, Pantoea, and Erwinia.

An animal host cell may include a mammalian host cell, for example, a fibroblast cell, an epithelial cell, a lymphocyte, or a macrophage. An animal host cell according to the present disclosure may be an immortalized animal cell line, a primary cell, or a stem cell.

In another example, the plant cell may be a dicotyledonous or a monocotyledonous plant cell, such as a plant cell selected from the group consisting of a Fabaceae, sunflower, safflower, sesame, tobacco, potato, cotton, sweet potato, cassava, coffee, tea, apple, pear, fig, citrus tree, cocoa, avocado, olive, almond, walnut, strawberry, watermelon, pepper, beet, grape, tomato, cucumber, thale cress, Brassica sp., pea, alfalfa, barrel clover, pigeon pea, guar, carob, fenugreek, soybean, common bean, cowpea, mung bean, lima bean, fava bean, lentil, peanut, licorice, chickpea, oil palm, coconut, banana, corn, barley, sorghum, rice, and wheat cell.

In another aspect, the instant disclosure provides methods for producing a plant comprising a genomic modification, the method comprising: (a) expressing the recombinant DNA molecule of claim 1 and a guide RNA compatible with the protein encoded by said recombinant DNA molecule in a plant cell; (b) introducing a modification into at least one target site in the plant cell genome; (c) identifying and selecting one or more plant cells of step (b) comprising said modification in said plant genome; and (d) regenerating at least one plant from at least one or more cells selected in step (c). In certain examples, the modification may be a substitution, an insertion, an inversion, a deletion, a duplication, and a combination thereof. In some embodiments, plants for use in the methods provided may be monocotyledonous plant, such as a barley, B. oleracea, wheat, or corn plant.

In another aspect, the instant disclosure provides methods for improving gene targeting using CRISPR-Cas12a gene editing in crops, comprising the steps of: expressing the recombinant DNA molecule comprising a polynucleotide sequence selected from the group consisting of: a sequence with at least 85 percent identity to any of SEQ ID NOs:1, 3, 5, 7, and 8; a sequence comprising SEQ ID NOs:1, 3, 5, 7, and 8; a fragment of any of SEQ ID NOs:1, 3, 5, 7, and 8; and/or a sequence encoding a protein having at least 85 percent identity to any of SEQ ID NOs: 2, 4, 6, and 9; and a guide RNA compatible with the protein encoded by said recombinant DNA molecule in a plant cell; and/or introducing a modification into at least one target site in the plant cell genome; wherein said modification is introduced at a higher rate when compared to the rate of introduction of a modification using a method comprising expressing a DNA molecule encoding the amino acid of SEQ ID NO:46. In some embodiments, the sequence has at least 90 percent identity to any of SEQ ID NOs:1, 3, 5, 7, and 8 and encodes a protein having a modification at amino acid position 156 as compared to a protein comprising the amino acid sequence of SEQ ID NO:46. In some embodiments, the sequence has at least 95 percent identity to any of SEQ ID NOs:1, 3, 5, 7, and 8 and encodes a protein having a modification at amino acid position 156 as compared to a protein comprising the amino acid sequence of SEQ ID NO:46. In some embodiments, the sequence comprises any of SEQ ID NOs:1, 3, 5, 7, and 8. In some embodiments, the modification at amino acid position 156 is further defined as an aspartate to arginine substitution. In some embodiments, the polynucleotide sequence further comprises intron sequences of SEQ ID NOs:10-17.

Further provided are methods of producing progeny seed comprising the recombinant DNA molecules described herein, the method comprising: (a) planting a first seed comprising the recombinant DNA molecule of claim 1; (b) growing a plant from the seed of step (a); and (c) harvesting the progeny seed from the plants, wherein said harvested seed comprises said recombinant DNA molecule.

In yet another aspect, the present disclosure provides methods for introducing a genomic modification in a plant, said method comprising: (a) expressing a protein or fragment thereof encoded by the DNA molecules provided herein in a plant; and (b) expressing a guide RNA compatible with said protein or fragment thereof having nuclease activity in a plant cell.

The present disclosure further provides methods of detecting the presence of the recombinant DNA molecules provided herein in a sample comprising plant genomic DNA, comprising: (a) contacting said sample with a DNA probe that hybridizes under stringent hybridization conditions with genomic DNA from a plant comprising the recombinant nucleic DNAs, and does not hybridize under such hybridization conditions with genomic DNA from an otherwise isogenic plant that does not comprise the recombinant DNA molecule, wherein said probe is homologous or complementary to a fragment of any of SEQ ID NOs:1, 3, 5, 7, 8; or a sequence that encodes a protein comprising an amino acid sequence having at least 85%, or 90%, or 95%, or 98% or 99%, or about 100% amino acid sequence identity to any of SEQ ID NOs: 2, 4, 6, and 9; (b) subjecting said sample and said probe to stringent hybridization conditions; and (c) detecting hybridization of said DNA probe with said recombinant DNA molecule.

In another aspect, the present disclosure provides methods of detecting the presence of a nuclease protein, or fragment thereof, in a sample comprising protein, wherein said protein comprises the amino acid sequence of any of SEQ ID NOs: 2, 4, 6, and 9; or said protein comprises an amino acid sequence having at least 85%, or 90%, or 95%, or 98% or 99%, or about 100% amino acid sequence identity to any of SEQ ID NOs: 2, 4, 6, and 9; comprising: (a) contacting said sample with an immunoreactive antibody; and (b) detecting the presence of said protein, or fragment thereof.

In additional embodiments methods for modifying a polynucleotide segment encoding a Cas12a protein or fragment thereof having nuclease activity are provided, the methods comprising: (a) obtaining a polynucleotide sequence of any of SEQ ID NOs:1, 3, 5, 7, and 8; and (b) introducing a modification into at least one target site in the polynucleotide sequence such that the protein encoded by said polynucleotide sequence comprises a modification at amino acid position 156 as compared to a protein comprising the amino acid sequence of SEQ ID NO: 46. In these methods, the protein encoded by the modified polynucleotide sequence comprises an aspartate to arginine substitution at amino acid position 156 as compared to a polynucleotide segment lacking said modification. The modified polynucleotide sequence further comprises at least one intron sequence of any of SEQ ID NOs:10-17, or may comprise one or more intron sequences of any of SEQ ID NOs: 10-17. In further examples, the modified polynucleotide sequence comprises an aspartate to arginine modification at amino acid position 156 and further comprises at least one intron sequence of SEQ ID NOs:10-17.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a schematic representation of editing construct architectures tested in barley. Briefly, P-ZmUbi refers to the maize ubiquitin promoter; Cas12a refers to the LbCas12a CDS; T-Nos refers to the nopaline synthase terminator; TaU6 refers to the wheat U6 promoter; TaU3 refers to the wheat U3 promoter; DR refers to direct repeat crRNA; HH/HDV refers to ribozyme sequences; t refers to the poly-T terminator; V1 refers to the V1 array. V2 refers to the V2 array. Thick black arrows show the direction of transcription.

FIG. 2 shows the efficiency of targeting the HORVU.MOREX.r31HG0069960 gene using the V1 guide array with different LbCas12a constructs. Os refers to OsCas12a; Hs refers to HsCas12a; ttHs refers to ttHsCas12a; ttAt refers to ttAtCas12a; ttAt+int refers to ttAtCas12a+int. Blue bars show the number of T0 lines. Orange bars show a number of T0 lines containing targeted mutations.

FIG. 3 shows the results of five barley genes each targeted with ttHsCas12a using the V1 array in comparison to the V2 array. Blue bars show the % T0 V1 lines containing targeted mutations. Orange bars show % T0 V2 lines containing targeted mutations. The x-axis indicates the array guide order. Gene identifiers are shown.

FIG. 4 shows a representative phenotypic comparison of Golden promise having the wild-type 2 row phenotype as compared to Golden promise T0 plant mutated in HORVU.MOREX.r3.2HG0184740 showing 6 row phenotype.

FIG. 5 shows sequencing analysis of the HORVU.MOREX.r3.1HG0069960 gene in a representative barley line. Top: Amplicon sequencing revealed the presence of two alleles (−3 bp; TTTGGTGCTGCACAATGAAAGCAGACGGC; SEQ ID NO: 50; and −10 bp; TTTGGTGCTGCACAACAACAACTGAAAGCAGACGGC; SEQ ID NO: 51) in the primary T0 generation. Bottom: In T-DNA free T1 progeny, the same two alleles were identified, establishing inheritance of mutations. The bottom left panel shows the unedited sequence (TTTGGTGCTGCACAATGTCAACAACTGAAAGCAGACGGC; SEQ ID NO: 52) along the top compared with the sequence of the T1 homozygous 3 bp deletion (SEQ ID NO: 50). The bottom middle panel shows the unedited sequence (SEQ ID NO: 52) along the top compared with the T1 homozygous 10 bp deletion (SEQ ID NO: 51). The bottom right panel shows the unedited sequence (SEQ ID NO: 52) along the top compared with the sequence of the T1 heterozygote (GTTGATGGTTGGTGTTGGGCAATGCCCAATGAAAGCAGACGGC; SEQ ID NO: 53).

FIG. 6A shows a schematic representation of editing construct architectures tested in B. oleracea. Briefly, Nos refers to nopaline synthase terminator; Npt refers to neomycin phosphotransferase (conferring kanamycin resistance for bacterial selection of plasmids); 35S refers to cauliflower mosaic virus 35S promoter; E9 refers to rbc-E9 terminator (from Pisum sativum); ttAtCas12a refers to Arabidopsis codon optimized LbCas12a carrying the D156R “temperature tolerant” mutation; ttHsCas12a refers to Homo sapiens codon optimized LbCas12a coding sequence carrying the “temperature tolerant” D156R mutation; ttAtCas12a+int refers to Arabidopsis codon optimized LbCas12a carrying the D156R “temperature tolerant” mutation and eight Arabidopsis introns; Ubi10 refers to Arabidopsis ubiquitin 10 promoter U6 refers to Arabidopsis U626 promoter; HH/HDV refers to ribozyme sequences; DR refers to direct repeat crRNA; G_A, _B, _C, and _D refer to protospacers A, B, C & D; t refers to the poly-T terminator.

FIG. 6B shows a comparison of mutagenesis efficiencies of LbCas12a constructs S5, S6, S7, and S8 targeting Bo2g016480. A comparison of S5, S6, S7, and S8 is possible at target C where the respective efficiencies were 3%, 50%, 50%, and 68%.

FIG. 7 shows sequencing analysis of the Bo2g016480 gene in T-DNA free T1 B. oleracea plants. −3 bp, −9 bp & −12 bp alleles were revealed, establishing inheritance of mutations. The left panel shows the unedited sequence GAGTTTTGGTATGCAGATCAACATTATAAGAATGTACC (SEQ ID NO: 54) along the top compared with the sequence of the T1 homozygous 3 bp deletion (GAGTTTTGGTATGCAGATCAACATAAGAATGTACC; SEQ ID NO: 55). The middle panel shows the unedited sequence (SEQ ID NO: 54) along the top compared with the sequence of the T1 homozygous 9 bp deletion (GAGTTTTGGTATGCAGATCAACATGTACC; SEQ ID NO: 56). The right panel shows the unedited sequence (SEQ ID NO: 54) along the top compared with the sequence of the T1 homozygous 12 bp deletion (GAGTTTTGGTATGCAGATCAAGTACC; SEQ ID NO: 57).

FIG. 8 shows the universal genetic code chart showing all possible mRNA triplet codons (where T in the DNA molecule is replaced by U in the RNA molecule) and the amino acid encoded by each codon.

FIG. 9 shows construct architecture for evaluating gene editing efficiency of the ttHsCas12a and ttAtCas12a+8introns nucleases in wheat.

FIG. 10 shows construct architecture for evaluating gene editing efficiency of the ttAtCas12a+8introns nuclease in wheat.

FIG. 11 shows construct architecture for evaluating gene editing efficiency of ttAtCas12a nuclease with and without introns in Arabidopsis thaliana.

FIG. 12 shows additional construct architectures for evaluating gene editing efficiency of Cas12a variants in barley.

FIG. 13 shows construct architecture for 12 LbCas12a coding sequence variants.

### BRIEF DESCRIPTION OF THE SEQUENCES

SEQ ID NO:1 is the polynucleotide sequence of the Lachnospiraceae bacterium Cas12a gene, codon optimized for expression in Oryza sativa (OsCas12a).

SEQ ID NO:2 is the amino acid sequence of the Lachnospiraceae bacterium Cas12a protein, encoded by SEQ ID NO: 1 (OsCas12a).

SEQ ID NO:3 is the polynucleotide sequence of the Lachnospiraceae bacterium Cas12a gene, codon optimized for expression in Homo sapiens (HsCas12a).

SEQ ID NO:4 is the amino acid sequence of the Lachnospiraceae bacterium Cas12a protein, encoded by SEQ ID NO: 3 (HsCas12a).

SEQ ID NO:5 is the polynucleotide sequence of the Lachnospiraceae bacterium Cas12a gene, codon optimized for expression in Homo sapiens and encoding a protein with a D156R mutation compared with the wildtype Cas12a protein (ttHsCas12a).

SEQ ID NO:6 is the amino acid sequence of the Lachnospiraceae bacterium Cas12a protein, encoded by SEQ ID NO:5 (ttHsCas12a).

SEQ ID NO:7 is the polynucleotide sequence of the Lachnospiraceae bacterium Cas12a gene, codon optimized for expression in Arabidopsis and encoding a protein with a D156R mutation compared with the wildtype Cas12a protein (ttAtCas12a).

SEQ ID NO:8 is the polynucleotide sequence of the Lachnospiraceae bacterium Cas12a gene, codon optimized for expression in Arabidopsis and encoding a protein with a D156R mutation compared with the wildtype Cas12a protein, and further comprising 8 intron sequences (ttAtCas12a+int).

SEQ ID NO:9 is the amino acid sequence of the Lachnospiraceae bacterium Cas12a protein, encoded by SEQ ID NOs:7 and 8 (ttAtCas12a and ttAtCas12a+int, respectively)

SEQ ID NOs:10-17 are the polynucleotide sequences of the introns within SEQ ID NO: 8.

SEQ ID NO:18 is the polynucleotide sequence of the polynucleotide sequences of the V1 guide RNA array construct.

SEQ ID NO:19 is the polynucleotide sequence of the polynucleotide sequences of the V2 guide RNA array constructs.

SEQ ID NO:20 is a polynucleotide sequence encoding a guide RNA targeting the barley gene HORVU.MOREX.r3.1HG0069960.

SEQ ID NO:21 is a polynucleotide sequence encoding a guide RNA targeting the barley gene HORVU.MOREX.r3.1HG0069960.

SEQ ID NO:22 is a polynucleotide sequence encoding a guide RNA targeting the barley gene HORVU.MOREX.r3.1HG0069960.

SEQ ID NO:23 is a polynucleotide sequence encoding a guide RNA targeting the barley gene HORVU.MOREX.r3.1HG0069960.

SEQ ID NO:24 is a polynucleotide sequence encoding a guide RNA targeting the barley gene HORVU.MOREX.r3.2HG0184740.

SEQ ID NO:25 is a polynucleotide sequence encoding a guide RNA targeting the barley gene HORVU.MOREX.r3.2HG0184740.

SEQ ID NO:26 is a polynucleotide sequence encoding a guide RNA targeting the barley gene HORVU.MOREX.r3.2HG0184740.

SEQ ID NO:27 is a polynucleotide sequence encoding a guide RNA targeting the barley gene HORVU.MOREX.r3.2HG0184740.

SEQ ID NO:28 is a polynucleotide sequence encoding a guide RNA targeting the barley gene HORVU.MOREX.r3.6HG0611290.

SEQ ID NO:29 is a polynucleotide sequence encoding a guide RNA targeting the barley gene HORVU.MOREX.r3.6HG0611290.

SEQ ID NO:30 is a polynucleotide sequence encoding a guide RNA targeting the barley gene HORVU.MOREX.r3.6HG0611290.

SEQ ID NO:31 is a polynucleotide sequence encoding a guide RNA targeting the barley gene HORVU.MOREX.r3.6HG0611290.

SEQ ID NO:32 is a polynucleotide sequence encoding a guide RNA targeting the barley gene HORVU.MOREX.r3.7HG0640970.

SEQ ID NO:33 is a polynucleotide sequence encoding a guide RNA targeting the barley gene HORVU.MOREX.r3.7HG0640970.

SEQ ID NO:34 is a polynucleotide sequence encoding a guide RNA targeting the barley gene HORVU.MOREX.r3.7HG0640970.

SEQ ID NO:35 is a polynucleotide sequence encoding a guide RNA targeting the barley gene HORVU.MOREX.r3.7HG0640970.

SEQ ID NO:36 is a polynucleotide sequence encoding a guide RNA targeting the barley gene HORVU.MOREX.r3.2HG0133680.

SEQ ID NO:37 is a polynucleotide sequence encoding a guide RNA targeting the barley gene HORVU.MOREX.r3.2HG0133680.

SEQ ID NO:38 is a polynucleotide sequence encoding a guide RNA targeting the barley gene HORVU.MOREX.r3.2HG0133680.

SEQ ID NO:39 is a polynucleotide sequence encoding a guide RNA targeting the barley gene HORVU.MOREX.r3.2HG0133680.

SEQ ID NO:40 is a polynucleotide sequence encoding an N-terminal nuclear localization signal.

SEQ ID NO:41 is the amino acid sequence of the N-terminal nuclear localization signal encoded by SEQ ID NO:40.

SEQ ID NO:42 is a polynucleotide sequence encoding a C-terminal nuclear localization signal, codon optimized for expression in Oryza sativa.

SEQ ID NO:43 is the amino acid sequence of the C-terminal nuclear localization signal, encoded by SEQ ID NOs:42, 44, and 45.

SEQ ID NO:44 is a polynucleotide sequence encoding a C-terminal nuclear localization signal, codon optimized for expression in Homo sapiens.

SEQ ID NO:45 is a polynucleotide sequence encoding a C-terminal nuclear localization signal, codon optimized for expression in Arabidopsis.

SEQ ID NO:46 is the amino acid sequence of the wild-type Lachnospiraceae bacterium Cas12a protein.

SEQ ID NO: 47 is a DNMT1 guide RNA sequence.

SEQ ID NO: 48 is a EMX1 guide RNA sequence.

SEQ ID NO: 49 is a FANCF guide RNA sequence.

SEQ ID NO: 50 is 3 bp deletion allele in a HORVU.MOREX.r3.1HG0069960 gene.

SEQ ID NO: 51 is a 10 bp deletion allele in a HORVU.MOREX.r3.1HG0069960 gene.

SEQ ID NO: 52 is an unedited allele in a HORVU.MOREX.r3.1HG0069960 gene.

SEQ ID NO: 53 is a sequence of the HORVU.MOREX.r3.1HG0069960 gene in the T1 heterozygote.

SEQ ID NO: 54 is an unedited allele in the Bo2g016480 gene.

SEQ ID NO: 55 is a 3 bp deletion allele in Bo2g016480 gene.

SEQ ID NO: 56 is a 9 bp deletion allele in Bo2g016480 gene.

SEQ ID NO: 57 is a 12 bp deletion allele in Bo2g016480 gene.

SEQ ID NO: 58 is a polynucleotide sequence encoding a Cas12a variant, codon optimized for expression in rice and comprising 12 introns (OsCas12a+12 introns).

### DETAILED DESCRIPTION

The clustered regularly interspaced short palindromic repeats (CRISPR)-Cas9 system represents the most widely used genome editing platform for targeted genome modifications in plants. For genome editing applications, a CRISPR/Cas9 system consists of two essential components: a Cas9 effector protein, which induces blunt-end (i.e., both DNA strands are of equal length) double strand breaks (DSBs), and a single-guide RNA (sgRNA), which contains an approximately 20 nt targeting sequence. DSBs are repaired primarily through either nonhomologous end joining (NHEJ) or homology-directed repair (HDR) pathways. Loss of function mutations are generated by short indels introduced during NHEJ-mediated repair pathway, whereas specific sequence modifications can be achieved by HDR pathway in the presence of a proper repair template, albeit at a much lower efficiency.

While the CRISPR-Cas9 system is still the most popular plant genome editing tool, the Lachnospiraceae bacterium CRISPR-Cas12a (LbCas12a) nuclease (originally identified as Cpf1) has also been shown to be capable of targeted genome modifications in plants. LbCas12a differs in its requirements and outcomes as compared to Streptococcus pyogenes Cas9 (SpCas9). Firstly, LbCas12a has a “TTTV” PAM sequence requirement making it useful in A-T rich regions, while SpCas9 requires “NGG” making it useful in G-C rich sequences. Secondly, SpCas9 typically results in indels of around 1-3 bp, whilst LbCas12a usually produces deletions of around 3-12 bp. Thirdly, SpCas9 cuts at the PAM proximal end of the target giving blunt ends, while LbCas12a cuts at the PAM distal region, giving sticky ends (i.e., one strand is longer than the other). LbCas12a's distinct PAM requirement, mutation profile, and DNA strand structure at the cleavage site all represent potential advantages in the field of precise genome editing and engineering in plants.

However, editing using SpCas9 and LbCas12a nucleases is not interchangeable; and modifications shown to increase Cas9 editing efficiency do not necessarily increase efficiency when the corresponding modification is made to Cas12a. Moreover, the current efficiency of editing using LbCas12a in various plant species, e.g., barley, B. oleracea, wheat, and corn is still extremely low (e.g. <10%). Thus, there is a continuing need for discovery and development of new strategies for increasing the efficiency of precise genome editing.

The present disclosure overcomes the limitations of the prior art by providing engineered Cas12a proteins, and the novel recombinant DNA molecules that encode them as well as compositions and methods using the same. The novel Cas12a variants are proteins having nuclease activity in a plant cell. The novel Cas12a variants yield significantly increased editing efficiencies in plants when used in combination with various guide RNA architectures as compared to control Cas12a proteins. One or more guide RNAs can be utilized. Guide RNAs known in the art (see e.g., Wang, 2021) can be selected by testing for mutagenesis of target genes. Transgenic plants expressing novel Cas12a sequences demonstrate improved genome editing efficiency for application in plant species widely known to exhibit low editing efficiencies using CRISPR-Cas9 as well as Cas12a editing techniques. Accordingly, provided herein are methods and compositions for targeted genome editing in plants that may be used to achieve beneficial results, including, e.g., improved reliability of producing edited plants, a significant increase in the number of edited T0 plants, an increase in the number T0 plants homozygous for a targeted edit, or combinations thereof. Moreover, the ability to produce these desirable characteristics in T0 plants with high efficiency offers unique benefits not otherwise available in the art.

To produce such plants, the present disclosure provides, in certain embodiments, methods, and compositions for the creation of targeted genome modification via the novel Cas12a sequences described herein. For example, a recombinant DNA molecule comprising a polynucleotide sequence encoding a Cas12a protein in combination with one or more guide RNAs was used to edit a plant genome as disclosed herein. For example, exemplary genes from two plant species known to exhibit low editing efficiencies, i.e., barley and B. oleracea, were targeted for mutagenesis. T0 plants transformed with the novel Cas12a sequences were selected and evaluated for editing efficiency and fidelity. It was shown that edited alleles at the target genes could be generated at significantly increased efficiencies compared to currently available methods. T0 plants both homozygous as well as heterozygous for the edited alleles were produced, and inheritance of the edited alleles was further identified in progeny plants (T1 plants). As described herein, novel Cas12a sequences using various gRNA architectures exhibited significant increases in editing efficiency in plant species known to exhibit low editing efficiencies using CRISPR-Cas genome editing techniques. The present disclosure thus represents a significant advance in the art in that it permits the production of engineered alleles in plants at high frequency.

### I. Engineered Proteins and Recombinant DNA Molecules

Provided herein are novel, engineered proteins and the recombinant DNA molecules that encode them. As used herein, a “Cas12a sequence,” “Cas12a variant,” or a protein having “nuclease activity” refers to a protein, specifically a Cas12a nuclease. As used herein, the term “engineered” refers to a non-natural DNA, protein, cell, or organism that would not normally be found in nature and was created by human intervention. An “engineered protein,” “engineered enzyme,” or “engineered nuclease,” refers to a protein, enzyme, or Cas12a nuclease whose amino acid sequence was conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene editing, gene cloning, DNA ligation, DNA synthesis, protein synthesis, and DNA shuffling. For example, an engineered protein may have one or more deletions, insertions, or substitutions relative to the coding sequence of the wild-type protein and each deletion, insertion, or substitution may consist of one or more amino acids. Genetic engineering can be used to create a DNA molecule encoding an engineered protein, such as an engineered Cas12a protein or Cas12a variant and comprises at least a first amino acid substitution relative to a wild-type Cas12a protein as described herein.

Examples of engineered proteins provided herein are RNA-guided Cas12a nucleases (referred to herein as “Cas12a proteins” or “Cas12a variants”) comprising at least 70% sequence identity to an amino acid sequence of SEQ ID NO:46, wherein the protein comprises at least one amino acid substitution as compared to SEQ ID NO:46. For example, wherein the protein comprises an arginine (R) at the position corresponding to position 156 of SEQ ID NO:46. In specific embodiments, an engineered protein provided herein comprises one, two, three, four, five, six, seven, eight, nine, ten, or more substitutions.

Engineered proteins are enzymes that have nuclease activity. As used herein, “nuclease activity” means the ability of a protein to introduce a double-stranded break (DSB) or single-stranded nick into the nucleic acid backbone of the polynucleotide sequence and/or its complementary DNA strand within the plant genome. Examples of proteins having nuclease activity include RNA-guided nucleases, such as Cas12a. Enzymatic activity of RNA-guided nucleases can be measured by any means known in the art, for example, by sequencing the genomic DNA within the target region of the RNA-guided nuclease following expression of said nuclease and at least of gRNA in a plant cell. In particular, RNA-guided nuclease activity can be identified based on the production of deletions of around 1-3 bp or 3-12 bp in the targeted genomic region.

The present disclosure provides a polynucleotide sequence encoding a protein having nuclease activity comprising at least 70% sequence identity to an amino acid sequence of SEQ ID NO:46, wherein the encoded protein comprises at least one amino acid substitution as compared to SEQ ID NO:46. For example, wherein the encoded protein comprises an arginine (R) at the position corresponding to position 156 of SEQ ID NO:46. In specific embodiments, an engineered protein provided herein comprises one, two, three, four, five, six, seven, eight, nine, ten, or more substitutions. Additionally, the present disclosure provides a polynucleotide sequence encoding a protein having nuclease activity comprising at least 85% sequence identity to a polynucleotide sequence of SEQ ID NO:46, wherein the protein encoded by said polynucleotide sequence comprises a modification at amino acid position 156 as compared to a protein comprising the amino acid sequence of SEQ ID NO:46. For example, wherein the protein comprises: an arginine (R) at the position corresponding to position 156 of SEQ ID NO:46. The present disclosure also provides a polynucleotide sequence encoding a protein having nuclease activity comprising at least 70% sequence identity to an amino acid sequence of SEQ ID NO:46, wherein said polynucleotide sequence further comprises at least one intron sequence of any of SEQ ID NOs:10-17. In some examples, polynucleotides of the present disclosure include at least one intron taken from an Arabidopsis gene The splicing efficiency of an intron from an Arabidopsis gene may be evaluated for inclusion in a polynucleotide of the present invention using bioinformatic methods such as the Netgene splicing tool (Hebsgaard, 1996) or alternatively through in vitro or in vivo assays, and one or more introns may be selected for inclusion in a polynucleotide of the present disclosure based on such methods. Methods of identifying introns in Arabidopsis have been described, (see, e.g., Cheng, 2018). In certain embodiments, said polynucleotide sequence encoding a protein having nuclease activity comprising at least 70% sequence identity to an amino acid sequence selected from the group consisting of SEQ ID NO:46 comprises an arginine (R) at the position corresponding to position 156 of SEQ ID NO:46, and said polynucleotide sequence further comprises at least one intron sequence for a plant, such as Arabidopsis, or of any of SEQ ID NOs:10-17, or a combination thereof.

As used herein, the term “protein-coding DNA molecule” or “a sequence encoding a protein” refers to a DNA molecule comprising a DNA sequence that encodes a protein. As used herein, the term “protein” refers to a chain of amino acids linked by peptide (amide) bonds and includes both polypeptide chains that are folded or arranged in a biologically functional way and polypeptide chains that are not. As used herein, a “protein-coding sequence” means a DNA sequence that encodes a protein. As used herein, a “sequence” means a sequential arrangement of nucleotides or amino acids. A “DNA sequence” may refer to a sequence of nucleotides or to the DNA molecule comprising of a sequence of nucleotides; a “protein sequence” may refer to a sequence of amino acids or to the protein comprising a sequence of amino acids. The boundaries of a protein-coding sequence are usually determined by a translation start codon at the 5′-terminus and a translation stop codon at the 3′-terminus.

Engineered proteins may be produced by changing or modifying a wild-type protein sequence to produce a new protein with modified characteristic(s) or a novel combination of useful protein characteristics, such as altered Vmax, Km, Ki, IC₅₀, substrate specificity, substrate selectivity, ability to interact with other components in the cell such as partner proteins or membranes, and protein stability, among others. Modifications may be made at specific amino acid positions in a protein and may be made by substituting an alternate amino acid for the typical amino acid found at that same position in nature (that is, in the wild-type protein). Amino acid modifications may be made as a single amino acid substitution in the protein sequence or in combination with one or more other modifications, such as one or more other amino acid substitution(s), deletions, or additions. In some embodiments, an engineered protein has altered protein characteristics, such as those that result in increased editing efficiency in the presence of one or more gRNA sequences as compared to the wild-type protein in the presence of the same gRNA sequences. In other embodiments, the present disclosure therefore provides an engineered protein such as a Cas12a variant, and the recombinant DNA molecule encoding it, having one or more amino acid substitution(s), e.g. D156R, wherein the position of the amino acid substitution(s) is relative to the amino acid position set forth in SEQ ID NO:46. In specific embodiments, an engineered protein provided herein comprises one, two, three, four, five, six, seven, eight, nine, ten, or more of any combination of such substitutions, wherein the modification is made at a position relative to a position comparable in function to that in the amino acid sequence provided as SEQ ID NO:46. Similar modifications can be made in analogous positions of any RNA-guided nucleases by alignment of the amino acid sequence of the RNA-guided nucleases to be mutated with the amino acid sequence of RNA-guided nucleases of interest that has nuclease activity e.g. Cas12a.

Any number of methods well known to those skilled in the art can be used to isolate and manipulate a DNA molecule, or fragment thereof, as disclosed herein. For example, polymerase chain reaction (PCR) technology can be used to amplify a particular starting DNA molecule or to produce variants of the original molecule. DNA molecules, or fragment thereof, can also be obtained by other techniques, such as by directly synthesizing the fragment by chemical means, as is commonly practiced by using an automated oligonucleotide synthesizer.

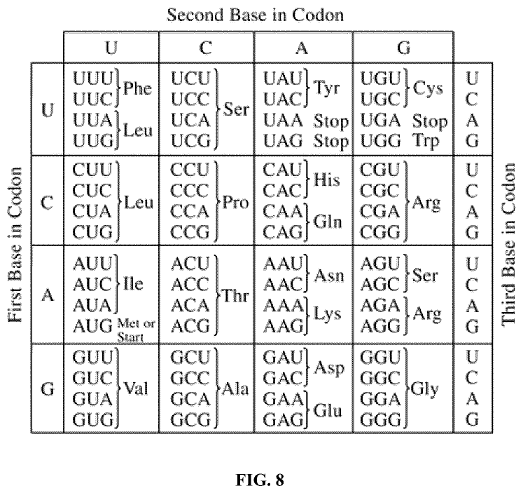

Because of the degeneracy of the genetic code, a variety of different DNA sequences can encode proteins, such as the altered or engineered proteins disclosed herein. For example, FIG. 8 provides the universal genetic code chart showing all possible mRNA triplet codons (where T in the DNA molecule is replaced by U in the RNA molecule), and the amino acid encoded by each codon. DNA sequences encoding Cas12a proteins with the amino acid substitutions described herein can be produced by introducing mutations into the DNA sequence encoding a wild-type Cas12a protein using methods known in the art and the information provided in FIG. 8. It is well within the capability of one of skill in the art to create alternative DNA sequences encoding the same, or essentially the same, altered or engineered proteins as described herein. These variant or alternative DNA sequences are within the scope of the embodiments described herein. As used herein, references to “essentially the same” sequence refers to sequences which encode amino acid substitutions, deletions, additions, or insertions that do not materially alter the functional activity (i.e., alter the function) of the protein encoded by the DNA molecule of the embodiments described herein. Allelic variants of the nucleotide sequences encoding a wild-type or engineered protein are also encompassed within the scope of the embodiments described herein. While maintaining the functional activity of the protein encoded by the DNA molecule, such allelic variants may produce beneficial effects when expressed in certain plant cells. For example, the results described herein demonstrate that Cas12a proteins and variants thereof, codon optimized for distantly related plant species or species in separate biological kingdoms, surprisingly resulted in increased genomic editing efficiency in plant species known to be recalcitrant to CRISPR-Cas genome editing, e.g., barley, B. oleracea, wheat, and corn.

Substitution of amino acids other than those specifically exemplified or naturally present in a wild-type or engineered Cas12a protein are also contemplated within the scope of the embodiments described herein, so long as the Cas12a protein having the substitution still retains substantially the same functional activity described herein. These variant or alternative DNA sequences in combination with such amino acid substitutions in the protein encoded by the DNA sequence are also encompassed within the scope of the embodiments described herein, including, but not limited to, SEQ ID NOs:1, 3, 5, 7, and 8. Similarly, variant or alternative DNA sequences encoding a Cas12a protein having nuclease activity further comprising heterologous intron sequences are also encompassed within the scope of the embodiments described herein. Introns do not contain information coding for a protein or polypeptide. Introns are first transcribed into an RNA sequence, but then spliced out from a mature RNA molecule. While maintaining the functional activity of the protein encoded by the DNA molecule further comprising heterologous intron sequences, such allelic variants comprising intron sequences may produce beneficial effects when expressed in certain plant cells.

For example, the results described herein demonstrate that Cas12a proteins and variants thereof, comprising at least one intron sequence of any of SEQ ID NOs:10-17 resulted in increased genomic editing efficiency in plant species known to exhibit low editing efficiencies using CRISPR-Cas genome editing techniques, e.g., barley, B. oleracea, wheat, and corn.

Polynucleotide sequences encoding Cas12a nucleases provided herein include polynucleotide sequences comprising at least 1, at least 2, at least 3, at least 4, at least 5, at least 6, at least 7, at least 8, at least 9, at least 10, at least 11, at least 12, at least 13, at least 14, at least 15, or more, intron sequences. Intron sequences which may be inserted into polynucleotide sequences encoding a Cas12a nuclease include, but are not limited to, any of SEQ ID NOs: 10-17, or multiple copies thereof. According to the present disclosure, an intron or introns may be inserted at any position within a sequence encoding a Cas12a nuclease, for example at any position within any of SEQ ID NOs: 1, 3, 5, 7, and 8. Experiments can be performed that can measure the combinatorial effect of the D156R mutation and the inclusion of one or more introns (e.g., comparing just a first intron compared with having any other or all eight introns in Cas12a). Other experiments can determine the portions of the Cas12a that contain introns that result in increased editing efficiency.

Recombinant DNA molecules provided herein may be synthesized and modified by methods known in the art, either completely or in part, where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). The present disclosure includes recombinant DNA molecules and engineered proteins having at least 50% sequence identity, at least 60% sequence identity, at least 70% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, and at least 99% sequence identity to any of the recombinant DNA molecule or amino acid sequences provided herein, and having nuclease activity. As used herein, the term “percent sequence identity” or “% sequence identity” refers to the percentage of identical nucleotides or amino acids in a linear polynucleotide or amino acid sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar Inc., 1228 S. Park St., Madison, WI 53715), and MUSCLE (version 3.6) (RC Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

### II. Genome Editing

The present disclosure provides, in certain embodiments, plants, plant parts, plant cells, and seeds produced through genome modification using site-specific integration or genome editing. Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 or Cas12a system), a TALE (transcription activator-like effector)-endonuclease (TALEN), a recombinase, or a transposase. In particular embodiments, a “targeted genome editing technique” refers to an RNA-guided Cas12a system. As used herein, “editing” or “genome editing” refers to generating a targeted mutation, deletion, insertion, substitution, inversion or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

According to some embodiments, a site-specific nuclease may be co-delivered with a donor template molecule to serve as a template for making a desired edit, mutation, or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 (also referred to herein as Cas12a) enzyme. Furthermore, in some embodiments, the RNA-guided endonuclease is a Cas12a enzyme or variant. In particular embodiments, the RNA-guided endonuclease is a Lachnospiraceae bacterium Cas12a (LbCas12a) variant encoded by a sequence with at least 85 percent identity to any of SEQ ID NOs:1, 3, 5, 7, and 8. The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. As described herein, the guide RNA may be transformed or introduced into a plant cell or tissue as a gRNA molecule, or as a recombinant DNA molecule, construct or vector comprising a transcribable DNA sequence encoding one or more guide RNAs operably linked to a single promoter or individual promoters. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). In comparison, the CRISPR-Cas12a system does not require a trans-activating crispr RNA (tracrRNA) for biogenesis of mature crRNA. Instead, the RuvC endonuclease domain of Cas12a processes its mature crRNA directly. A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As mentioned above, a target gene for genome editing may be any plant gene of interest. For knockdown mutations of the gene of interest through genome editing, an RNA-guided endonuclease may be targeted to an upstream or downstream sequence, such as a promoter and/or enhancer sequence, or an intron, 5′UTR, and/or 3′UTR sequence of the gene to mutate one or more promoter and/or regulatory sequences of the gene to affect or reduce its level of expression. Similarly, mutations of the gene of interest through genome editing, an RNA-guided endonuclease may be targeted to a transcribable DNA sequence (i.e., a transcribable region) of said gene, such as a region of the gene comprising a coding sequence, a specific DNA sequence encoding a protein domain, an exon region, an intron region, or a combination thereof. For example, in certain embodiments a transcribable DNA sequence targeted for genome editing may comprise an exon/intron boundary or may be in close proximity to an exon/intron boundary. If the resulting modification spans an exon/intron boundary, the modification may be referred to as a modification in an exon region and an intron region. For genetic modification of the gene of interest, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of said gene or a sequence complementary thereto, although alternative splicing and different exon/intron boundaries may occur. As used herein, the term “consecutive” in reference to a polynucleotide or protein sequence means without deletions or gaps in the sequence.

As used herein, respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs. A ribosomal skipping sequence (e.g., 2A sequence such as furin-GSG-T2A) can be used in a construct to prevent covalently linking translated amino acid sequences.

tRNA an alternate guide architecture incorporating tRNA sequences instead of ribozymes, can also be used. One or more tRNAs can be used.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2(2):59-70, 2014). The genomic PAM sequence on the sense (+) strand adjacent to the target site (relative to the targeting sequence of the guide RNA) may comprise 5′-NGG-3′ for Cas9; or 5′-TTTN-3′ for Cas12a. However, the corresponding sequence of the guide RNA (i.e., immediately downstream (3′) to the targeting sequence of the guide RNA) may generally not be complementary to the genomic PAM sequence.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes, or transcribable DNA sequences.

Without being limited by example, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, one or more exon(s), one or more intron(s), transcriptional termination site, region, or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct, or sequence is located in the insertion sequence of the donor template.

The introduction of a DSB or nick may also be used to introduce targeted mutations in the genome of a plant. According to this approach, mutations, such as deletions, insertions, substitutions, inversions, and/or duplications may be introduced at a target site via imperfect repair of the DSB or nick to produce a genetic modification within a gene. Such mutations may be generated by imperfect repair of the targeted locus even without the use of a donor template molecule. A modification of a gene may be achieved by inducing a DSB or nick at or near the endogenous locus of the gene that results in expression of a non-functional protein, interfering protein, or a protein having reduced, disrupted, or altered activity as compared to a protein expressed from the gene lacking said modification.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking said modification.

In an aspect, the present disclosure provides a plant, or plant seed, plant part or plant cell thereof, comprising a recombinant DNA molecule, wherein the recombinant DNA molecule comprises a sequence with at least 85 percent identity to any of SEQ ID NOs:1, 3, 5, 7, and 8; a sequence comprising any of SEQ ID NOs:1, 3, 5, 7, and 8; a fragment of any of SEQ ID NOs:1, 3, 5, 7, and 8; or a sequence encoding a protein having at least 85 percent identity to any of SEQ ID NOs:2, 4, 6, and 9. In certain embodiments, the protein encoded by the recombinant DNA molecule comprises (i) a modification at amino acid position 156 as compared to a protein comprising the amino acid sequence of SEQ ID NO:46; (ii) further comprises one or more intron sequences of SEQ ID NOs:10-17; or a combination thereof. When expressed in a plant cell in the presence of one or more guide RNA molecules, the protein encoded by the recombinant DNA molecules described herein may yield genomic modifications within a target region defined by the gRNA(s) at high efficiency as compared to a control protein, e.g. as compared to a protein comprising the amino acid sequence of SEQ ID NO:46. The genome modification may be a deletion of a region comprising at least 1, at least 2, at least 4, at least 6, at least 8, at least 10, at least 20, at least 30, at least 40, at least 50, at least 60, at least 70, at least 75, at least 80, at least at least 90, at least 95, at least 100, or at least 150 consecutive nucleotides within the target region. In an aspect, the genome modification may also comprise a deletion and nucleotide substitutions or nucleotide insertions of at least 1, at least 2, at least 4, at least 6, at least 8, at least or at least 20 consecutive nucleotides around the deletion.

In an aspect, a mutant allele of the gene of interest may comprise two or more modifications in the transcribable region of the endogenous gene. The present disclosure provides for such mutant alleles, which may be produced, e.g., using a construct comprising a sequence encoding two or more guide RNAs operably linked to a plant expressible promoter; or a construct comprising two gRNA cassettes each operably linked to a plant expressible promoter.

### III. Constructs for Genome Editing

Recombinant DNA constructs and vectors are provided comprising a polynucleotide sequence encoding a site-specific nuclease, such as an RNA-guided endonuclease, wherein the coding sequence is operably linked to a plant expressible promoter. For RNA-guided endonucleases, recombinant DNA constructs and vectors are further provided comprising a polynucleotide sequence encoding one or more guide RNA(s), wherein the guide RNA(s) comprise a guide sequence of sufficient length having a percent identity or complementarity to a target site within the genome of a plant, such as at or near a targeted gene of interest. A polynucleotide sequence of a recombinant DNA construct and vector that encodes a site-specific nuclease or a guide RNA(s) may be operably linked to a plant expressible promoter, such as an inducible promoter, a constitutive promoter, a tissue-specific promoter, etc.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have reduced, disrupted, altered, or eliminated activity, or a reduced or eliminated expression level for the gene relative to the wild-type allele. For example, a mutant or edited allele for a gene of interest may have a deletion in the transcribable region of the endogenous gene that reduces, disrupts, or alters the activity of the protein encoded by the mutant allele as compared to the activity of the protein encoded by the wild-type allele in an otherwise identical plant. For diploid organisms, e.g., corn, a first allele can occur on one chromosome, and a second allele can occur at the same locus on a second homologous chromosome. If one allele at a locus on one chromosome of a plant is a mutant or edited allele and the other corresponding allele on the homologous chromosome of the plant is wild type, then the plant is described as being heterozygous for the mutant or edited allele. However, if both alleles at a locus are mutant or edited alleles, then the plant is described as being homozygous for the mutant or edited alleles. A plant homozygous for mutant or edited alleles at a locus may comprise the same mutant or edited allele or different mutant or edited alleles if heteroallelic or biallelic.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype. In general, the term “variant” refers to molecules with some differences, generated synthetically or naturally, in their nucleotide or amino acid sequences as compared to reference (native) polynucleotides or polypeptides, respectively. These differences include substitutions, insertions, deletions, inversions, duplications, or any desired combinations of such changes in a native polynucleotide or amino acid sequence.

As used herein, the term “expression” refers to the biosynthesis of a gene product, and typically the transcription and/or translation of a nucleotide sequence, such as an endogenous gene, a heterologous gene, a transgene, or an RNA and/or protein coding sequence, in a cell, tissue, organ, or organism, such as a plant, plant part or plant cell, tissue, or organ.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc. The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence. Similarly, “heterologous” in reference to a coding sequence may refer to the use of a recombinant DNA molecule codon-optimized for a different organism as compared to the organism said DNA molecule is being expressed in—e.g., the recombinant DNA sequence encoding a Cas12a is codon-optimized for expression in humans but is expressed in a plant cell.

As used herein, an “endogenous gene” or an “endogenous locus” refers to a gene or locus at its natural and original chromosomal location. As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into an RNA sequence but then spliced out from a mature RNA molecule.

As used herein, an “untranslated region (UTR)” of a gene refers to a segment of an RNA molecule or sequence (e.g., a mRNA molecule) expressed from a gene (or transgene), but excluding the exon and intron sequences of the RNA molecule. An “untranslated region (UTR)” also refers to a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcribable region” or “transcribable DNA sequence” refers to a nucleic acid sequence expressed from a gene (or transgene).

As used herein, a “transcription termination sequence” refers to a nucleic acid sequence containing a signal that triggers the release of a newly synthesized transcript RNA molecule from an RNA polymerase complex and marks the end of transcription of a gene or locus.

The terms “percent identity,” “% identity,” or “percent identical,” as used herein in reference to two or more nucleotide or protein sequences, is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When a percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process, orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing, or regenerating transformed explants, tissues, plants and/or plant parts.

### IV. Transformation Methods

Methods and compositions are provided for transforming a plant cell, tissue or explant with a recombinant DNA molecule or construct encoding one or more molecules required for targeted genome editing (e.g., guide RNA(s) and/or site-directed nuclease(s)). Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, Hinchliffe and Harwood (2019), and Sparrow and Irwin (2015). Other methods for plant transformation, such as microinjection, electroporation, vacuum infiltration, pressure, sonication, silicon carbide fiber agitation, PEG-mediated transformation, etc., are also known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores, and the like. Cells containing a transgenic nucleus are grown into transgenic plants. Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed, or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants.

According to an aspect of the present disclosure, regenerated plants or a progeny plant, plant part, or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait, or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote, and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations, and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Recombinant DNA molecules provided herein may be present within a host cell, wherein said host cell is any type of cell. Host cells contemplated by the present disclosure include cells selected from the group consisting of a bacterial cell, an animal cell, a plant cell, a yeast cell, a fugal cell, and an insect cell.

For example, a bacterial host cell that may be transformed with a recombinant DNA molecule or transformation vector comprising a Cas12a, guide RNA(s), or combination thereof, may be from a genus of bacteria selected from the group consisting of: Agrobacterium, Rhizobium, Bacillus, Brevibacillus, Escherichia, Pseudomonas, Klebsiella, Pantoea, and Erwinia.

An animal host cell that may be transformed with a recombinant DNA molecule or transformation vector comprising a Cas12a, guide RNA(s), or combination thereof, may include a mammalian host cell, for example a fibroblast cell, an epithelial cell, a lymphocyte, or a macrophage. An animal host cell according to the present disclosure may be an immortalized animal cell line, a primary cell, or a stem cell.

A plant cell that may be transformed with a recombinant DNA molecule or transformation vector comprising a Cas12a, guide RNA(s), or combination thereof, may include a variety of flowering plants or angiosperms, which may be further defined as including various dicotyledonous (dicot) plant species or monocotyledonous (monocot) plant species. A dicot plant could be members of the Fabaceae family (such as legumes), sunflower (Helianthus annuus), safflower (Carthamus tinctorius), sesame (Sesamum spp.), tobacco (Nicotiana tabacum), potato (Solanum tuberosum), cotton (Gossypium barbadense, Gossypium hirsutum), sweet potato (Ipomoea batatas), cassava (Manihot esculenta), coffee (Coffea spp.), tea (Camellia spp.), fruit trees, such as apple (Malus spp.), Prunus spp., such as plum, apricot, peach, cherry, etc., pear (Pyrus spp.), fig (Ficus carica), etc., citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), beet (Beta vulgaris), grape (Vitis, Muscadinia), tomato (Lycopersicon esculentum, Solanum lycopersicum), cucumber (Cucumis sativus), and members of the Brassicaceae family, such as thale cress (Arabidopsis thaliana) and Brassica sp. (e.g., B. napus, B. rapa, B. juncea), particularly those Brassica species useful as sources of seed oil. Legumes and leguminous plants include peas (Pisum sativum) alfalfa (Medicago sativa), barrel clover (Medicago truncatula), pigeon pea (Cajanus cajan) guar (Cyamopsis tetragonoloba), carob (Ceratonia siliqua), fenugreek (Trigonella foenum-graecum), soybean (Glycine max), common bean (Phaseolus vulgaris), cowpea (Vigna unguiculata), mung bean (Vigna radiata), lima bean (Phaseolus lunatus), fava bean (Vicia faba), lentil (Lens culinaris or Lens esculenta), peanut (Arachis hypogaea), licorice (Glycyrrhiza glabra), and chickpea (Cicer arietinum). A monocot plant could be oil palm (Elaeis spp.), coconut (Cocos spp.), banana (Musa spp.), and cereals such as corn (Zea mays), barley (Hordeum vulgare), sorghum (Sorghum bicolor), rice (Oryza sativa), and wheat (Triticum aestivum). Given that the present disclosure may apply to a broad range of plant species, the present disclosure further applies to other botanical structures analogous to pods of leguminous plants, such as bolls, siliques, fruits, nuts, tubers, etc.

### V. Genome Modified Plants

As used herein, “modified” in the context of a plant, plant seed, plant part, plant cell, and/or plant genome, refers to a plant, plant seed, plant part, plant cell, and/or plant genome comprising an engineered change in the expression level and/or sequence of one or more genes of interest relative to a wild-type or control plant, plant seed, plant part, plant cell, and/or plant genome. Indeed, the term “modified” may further refer to a plant, plant seed, plant part, plant cell, and/or plant genome having one or more deletions and/or one or more nucleotide substitutions or nucleotide insertions affecting an endogenous gene introduced through genome editing using any of the recombinant DNA molecules described herein. In an aspect, a modified plant, plant seed, plant part, plant cell, and/or plant genome can comprise one or more transgenes. For clarity, therefore, a modified plant, plant seed, plant part, plant cell, and/or plant genome includes a mutated, edited and/or transgenic plant, plant seed, plant part, plant cell, and/or plant genome having a modified genomic sequence relative to a wild-type or control plant, plant seed, plant part, plant cell, and/or plant genome.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the gene of interest. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence or edit. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s) (e.g., a deletion) affecting a gene of interest. For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s) affecting a gene of interest. Similarly, an “unmodified control plant” refers to a plant that shares a substantially similar or essentially identical genetic background as a modified plant, but without the one or more engineered changes to the genome (e.g., mutation or edit) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the terms “suppress,” “suppression,” “inhibit,” “inhibition,” “inhibiting,” “knockout,” “knockdown,” and “downregulation” refer to a lowering, reduction, or elimination of the expression level of an mRNA and/or protein encoded by a target gene in a plant, plant cell, or plant tissue at one or more stage(s) of plant development, as compared to the expression level of such target mRNA and/or protein in a wild-type or control plant, cell, or tissue at the same stage(s) of plant development.

As used herein, the term “activity” refers to the biological function of a gene or protein. A gene or a protein may provide one or more distinct functions. A reduction, disruption, or alteration in “activity” thus refers to a lowering, reduction, or elimination of one or more functions of a gene or a protein in a plant, plant cell, or plant tissue at one or more stage(s) of plant development, as compared to the activity of the gene or protein in a wild-type or control plant, cell, or tissue at the same stage(s) of plant development. Additionally, an increase in “activity” thus refers to an elevation of one or more functions of a gene or a protein in a plant, plant cell, or plant tissue at one or more stage(s) of plant development, as compared to the activity of the gene or protein in a wild-type or control plant, cell, or tissue at the same stage(s) of plant development.

According to some embodiments, a plant is provided having an mRNA level of a recombinant DNA molecule as described herein that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a plant is provided having an mRNA expression level of a recombinant DNA molecule as described herein that is reduced or increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a plant is provided having a protein expression level from a recombinant DNA molecule as described herein that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a plant is provided having a protein expression level from a recombinant DNA molecule as described herein that is reduced or increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

According to some embodiments, a plant is provided having an gRNA expression level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant.

According to some embodiments, a plant is provided having a recombinant DNA molecule that yields an increase in editing efficiency in at least one plant cell by at least 5%, at least 10%, at least 20%, at least 25%, at least 30%, at least 40%, at least 50%, at least 60%, at least 70%, at least 75%, at least 80%, at least 90%, or 100%, as compared to a control plant.

Modified plants comprising or derived from plant cells that comprise a genome modification of this disclosure can be further enhanced with stacked traits, for example, a modified crop plant having an enhanced trait resulting from expression of DNA disclosed herein in combination with one or more additional genome modifications that provide a beneficial agronomic trait or further improve the enhanced trait.

The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

Modified plants comprising or derived from plant cells that are transformed with a recombinant DNA of this disclosure can be further enhanced with stacked traits, for example, a modified crop plant having an enhanced trait resulting from expression of DNA disclosed herein in combination with one or more genes of agronomic interest that provide a beneficial agronomic trait (such as herbicide and/or pest resistance traits) to crop plants. For example, the traits conferred by the recombinant DNA constructs of the current disclosure can be stacked with other traits of agronomic interest, such as a trait providing insect resistance such as using a gene from Bacillus thuringensis to provide resistance against lepidopteran, coleopteran, homopteran, hemiopteran, and other insects, or improved quality traits such as improved nutritional value. Molecules and methods for imparting insect/nematode/virus resistance are disclosed in U.S. Pat. Nos. 5,250,515; 5,880,275; 6,506,599; 5,986,175; and U.S. Patent Application Publication No. 2003/0150017 A1.

### VI. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements. The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, seedling, or plantlet at any stage of regeneration or development.

As used herein, a “plant part” can refer to any organ or intact tissue of a plant, such as a meristem, shoot organ/structure (e.g., leaf, stem, or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

An “embryo” is a part of a plant seed, consisting of precursor tissues (e.g., meristematic tissue) that can develop into all or part of an adult plant. An “embryo” may further include a portion of a plant embryo.

A “meristem” or “meristematic tissue” comprises undifferentiated cells or meristematic cells, which are able to differentiate to produce one or more types of plant parts, tissues, or structures, such as all or part of a shoot, stem, root, leaf, seed, etc.

As used herein, “genomic DNA” or “gDNA” refers to chromosomal DNA of an organism. As used herein, a “genomic modification” (also referred to as “modification”) or “genomic edit” (also referred to as “edit”) refers to any modification to a genomic nucleotide sequence as compared to a wild-type or control plant. A genomic modification or genomic edit comprises a deletion, an insertion, a substitution, an inversion, a duplication, or any combination thereof.

As used herein, “T-DNA” or “transfer DNA” refers to the transferred DNA of the tumor-inducing (Ti) plasmid of some species of bacteria such as Agrobacterium tumefaciens.

As used herein, a “editing efficiency” (also referred to as “mutagenesis rate”) refers to the number of T0 lines containing a targeted mutation in comparison to the total number of T0 lines transformed with the applicable construct to produce the targeted mutation.

As used herein, the “vegetative phase” of plant development is the period of growth between germination and flowering. For maize, a common plant development scale used in the art is known as V-Stages. The V-stages are defined according to the uppermost leaf in which the leaf collar is visible. VE corresponds to emergence, V1 corresponds to first leaf, V2 corresponds to second leaf, V3 corresponds to third leaf, V(n) corresponds to nth leaf. VT occurs when the last branch of tassel is visible but before silks emerge. When staging a field of maize, each specific V-stage is defined only when 50 percent or more of the plants in the field are in or beyond that stage. Other development scales are known to those of skill in the art and may be used with the methods of the invention. The stages in the reproductive phase of maize are as follows R1 (silking; silks emerge from husks); R2 (blister; kernels are white on outside and inner fluid is clear); R3 (milk, kernels are yellow on the outside and inner fluid is milky-white); R4 (dough; milky inner fluid thickens from starch accumulation); R5 (dent; more than 50% of kernels are dented); and R6 (physiological maturity; black layer formed). Vegetative and reproductive stages for other agricultural crop species are well known to those of skill in the art and numerous publications describing these stages can be found on the world wide web and elsewhere.

As used herein, the term “isogenic” means genetically uniform, whereas non-isogenic means genetically distinct.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

### EXAMPLES

### Example 1. Evaluation of Novel Cas12a Variants with Single Promoter Guide Architecture in Barley

The editing efficiency of Lachnospiraceae bacterium Cas12a nuclease (LbCas12a) variants was evaluated in barley. In particular, a rice-optimized Cas12a coding sequence (CDS) (OsCas12a; SEQ ID NO:1), a human-optimized Cas12a CDS (HsCas12a; SEQ ID NO:3), functional in dicotyledonous plants, and an Arabidopsis-optimized Cas12a CDS containing the D156R “temperature tolerant” mutation (ttAtCas12a; SEQ ID NO:5) were chosen for evaluation. Two additional variants, HsCas12a carrying the D156R mutation (ttHsCas12a; SEQ ID NO:7) and ttAtCas12 carrying 8 introns (ttAtCas12+int; SEQ ID NO:8) were also created and evaluated. The constructs comprising the Cas12a nuclease variants selected for evaluation each further comprised a C-terminal nuclear localization signal operably linked to the respective codon optimized Cas12a nuclease variant. Briefly, OsCas12a comprised a polynucleotide of SEQ ID NO:42 (encoding SEQ ID NO:43); HsCas12a and ttHsCas12a comprised a polynucleotide of SEQ ID NO:44 (encoding SEQ ID NO:43); and ttAtCas12a and ttAtCas12+int comprised a polynucleotide of SEQ ID NO:45 (encoding SEQ ID NO:43). The OsCas12a variant further comprised an N-terminal nuclear localization signal (SEQ ID NO:40; encoding SEQ ID NO:41). The novel ttAtCas12a+int variant further comprises one synonymous G to A substitution at base 2471 to remove a cryptic splice site after intron insertion.

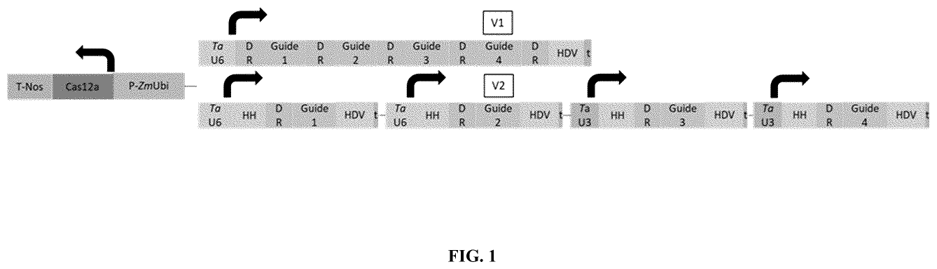

The target barley gene used in the evaluation was HORVU.MOREX.r3.1HG0069960 using the construct architecture shown in FIG. 1. A single U6 promoter was used to drive expression of 4 guide RNA sequences (SEQ ID NOs:20-23; also referred to herein as the V1 construct or V1 array). LbCas12a is able to process the single gRNA transcript containing multiple guides into individual guides by recognition of and cleavage at its own direct repeat (DR) sequence, which forms the invariable section of guides. A self-processing hepatitis delta ribozyme (HDV) sequence was placed at the 3′ end of the array prior to a terminator to prevent the formation of a spurious additional guide from the final DR. Five constructs each containing a single Cas12a nuclease (OsCas12a, HsCas12a, ttAtCas12a, ttHsCas12a, and ttAtCas12+int) and the same 4 gRNA sequences were created. The five constructs were individually transformed into barley cultivar Golden Promise using Agrobacterium mediated transformation and T0 plants were regenerated. DNA was extracted from T0 plants and the HORVU.MOREX.r3.1HG0069960 locus PCR amplified for sequencing analysis (Sanger sequencing). ABI files were analyzed by viewing chromatograms in alignments to wild type sequence using Benchling (https://www.benchling.com/) and targeted mutations were confirmed using the ICE tool (Synthego—CRISPR Performance Analysis) to score plants as either plus or minus for mutagenesis.

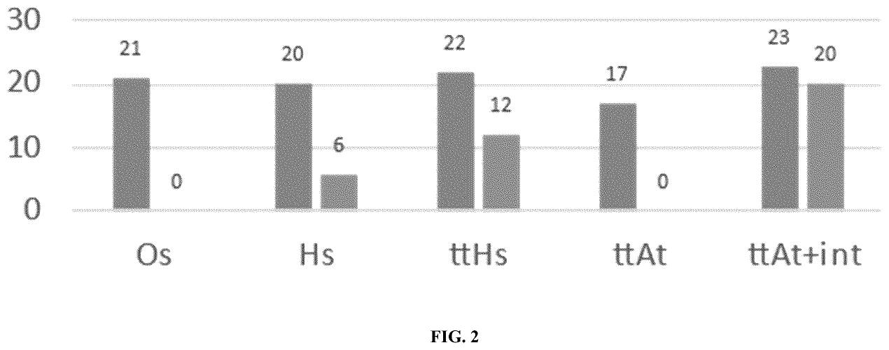

The number of T0 lines tested/containing mutations is shown in FIG. 2. Around 20 T0 lines were created for each of the five constructs which showed marked differences in the numbers of lines mutated at the target. The rice-optimized OsCas12a showed no mutated lines (0/21), while human-optimized HsCas12a gave 6/20 (30%) mutated lines. Interestingly, including the D156 mutation in the human-optimized sequence (ttHsCas12a) increased the mutation rate to 12/22 (54%). Even more interesting, the Arabidopsis-optimized Cas12a CDS containing the D156R “temperature tolerant” mutation (ttAtCas12a) gave no mutated lines (0/17) but adding introns (ttAtCas12a+int) gave 20/23 (87%) mutated lines. Thus, adding introns to the initially non-functional Arabidopsis CDS to give ttAtCas12+int transformed it into the most efficient CDS evaluated in barley. Moreover, the two novel LbCas12a variants, ttHsCas12a and ttAtCas12a+int both resulted in highly efficient targeted mutagenesis in barley. These results demonstrate the significant and surprising effect codon usage, the D156 mutation, and the presence of introns have on the efficiency of Cas12a mutagenesis in barley.

### Example 2. Evaluation of Novel Cas12a Variants with Multiple Promoter Guide Architecture in Barley

Although 4 gRNA sequences were used in the LbCas12a comparison described in Example 1, only two were determined to be active based on the sequencing results. To further verify the editing efficiency of the Cas12a variants described herein, constructs were evaluated using an additional gRNA construct, wherein each guide was driven by a separate TaU6/TaU3 promoter and flanked by self-cleaving ribozymes (also referred to herein as the V2 construct or V2 array); a 5′ Hammerhead (HH) and a 3′ HDV (Wolter 2019). Each HDV was followed by a transcription termination signal to prevent readthrough. This V2 construct was coupled with the ttHsCas12a and used to target HORVU.MOREX.r3.1HG0069960. Eight additional constructs (4 pairs) containing ttHsCas12a coupled with the V1 or V2 architecture were made, targeting four additional barley genes, each with 4 guide RNA sequences. This allowed direct comparison of V1/V2 guide architectures. Between 19 and 25 T0 lines were created for each construct that were PCR/Sanger sequenced, aligned, and ICE tested for targeted mutations as described in Example 1.

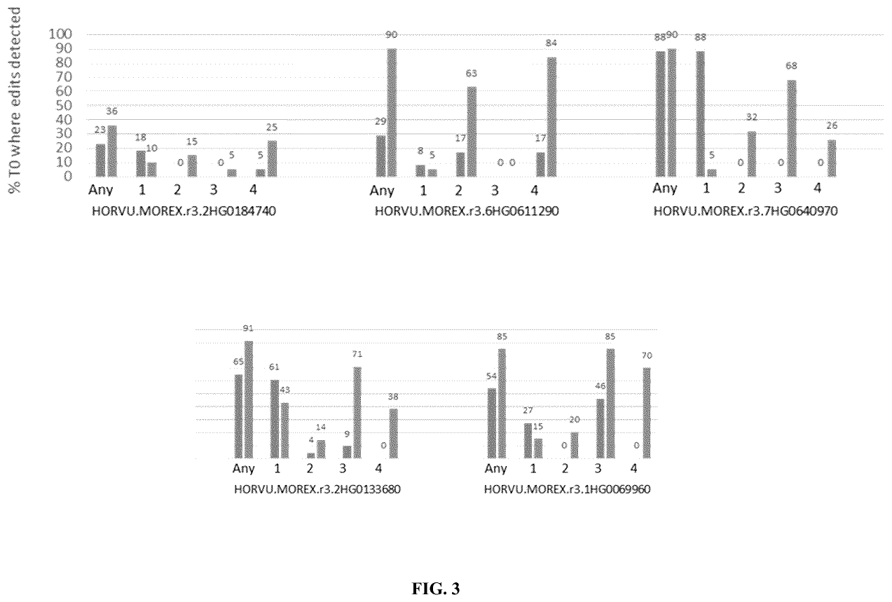

FIG. 3 shows the percentage of T0 lines carrying mutations at individual guide targets and the percentage of lines mutated at any guide targets. The V2 array was more efficient than the V1 array overall, giving the greatest percentage of T0 lines mutated at any guide target (36>23; 90>29; 90>88; 91>65; 85>54). Without being bound by any particular theory, the differences in editing efficiency when using the V1 array versus the V2 array may be attributable to varying abundances of the individual gRNAs. For example, the single TaU6 promoter may only transcribe short sequences, approximately equivalent in length to a single guide, such that downstream guides in array positions 2, 3 and 4 are underrepresented or absent. In V2 arrays, each of the 4 guides may be effectively transcribed due to transcription from its own promoter, making guide RNAs in array positions 1-4 abundant. In particular, V1 arrays showed higher mutagenesis with guides in array position 1 than V2 in array position 1 for all five target genes. Nonetheless, these results demonstrate that mutagenesis in around 90% of T0 plants for 4/5 barley target genes was achieved using ttHsCas12a with the V2 guide array. These results also indicate that editing efficiency in barley can be further increased using the ttAtCas12a+int variant, which performed best in the Cas12a comparison described in Example 1 (87%>54%).

### Example 3. Phenotypic Evaluation of Cas12a Variant Edited Barley and Inheritance of Edits in Progeny Plants

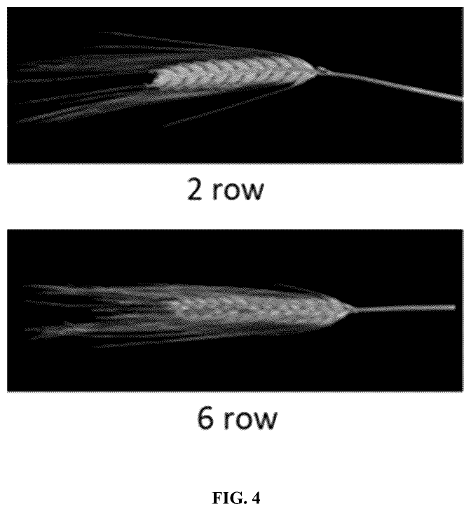

In order to investigate the ability of ttHsCas12a to yield knockout phenotypes in the first generation, the mutagenesis of barley gene HORVU.MOREX.r3.2HG0184740 was evaluated. Specifically, a construct comprising ttHsCas12a and a gRNA construct(s) targeting HORVU.MOREX.r3.2HG0184740 was transformed into barley cultivar Golden Promise using Agrobacterium mediated transformation as described in Examples 1 and 2. Knockout of both copies of HORVU.MOREX.r3.2HG0184740 is known to result in the conversion of two-rowed Golden Promise spikelets into six row spikelets (Komatsuda et al., 2007). This phenotype was seen in several active T0 lines when using both the V1 and V2 guide architecture. An example line comprising this phenotype is shown in FIG. 4. These results confirm that ttHsCas12a yielded the expected knockout phenotype in the first generation.

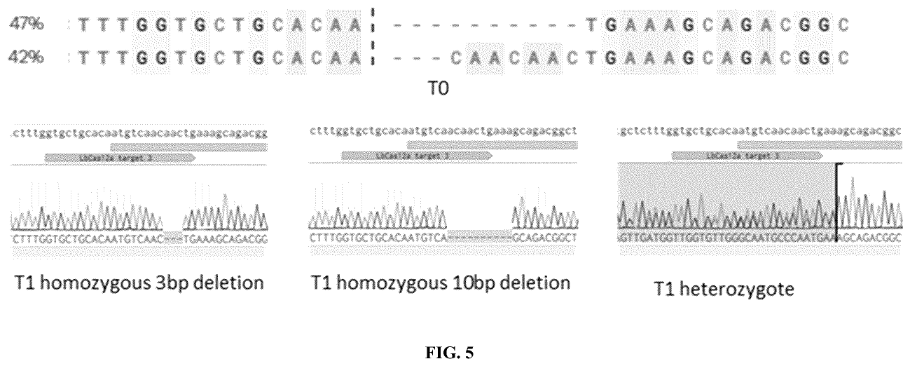

Further analysis of the T0 lines using the ICE tool, calculated one T0 line targeting HORVU.MOREX.r3.1HG0069960 contained 47% and 42% of −10 bp & −3 bp alleles respectively. Of 24 T1 plants produced therefrom, five were T-DNA free, of which two were homozygous for the 3 bp deletion, one was homozygous for the 10 bp deletion, and two were heterozygous (FIG. 5). These results demonstrate that mutations resulting from ttHsCas12a editing in T0 plants show inheritance in progeny plants.

### Example 4. Evaluation of Novel Cas12a Variants with Single and Multiple Promoter Guide Architectures in B. oleracea

The editing efficiency of Lachnospiraceae bacterium Cas12a nuclease (LbCas12a) variants was evaluated in B. oleracea. In particular, the human-optimized Cas12a CDS (HsCas12a), the Arabidopsis-optimized Cas12a CDS containing the D156R “temperature tolerant” mutation (ttAtCas12a), the novel HsCas12a carrying the D156R mutation (ttHsCas12a), and the ttAtCas12 carrying 8 introns (ttAtCas12+int) as described in Example 1 were chosen for evaluation. The target B. oleracea gene used in the evaluation was Bo2g016480.

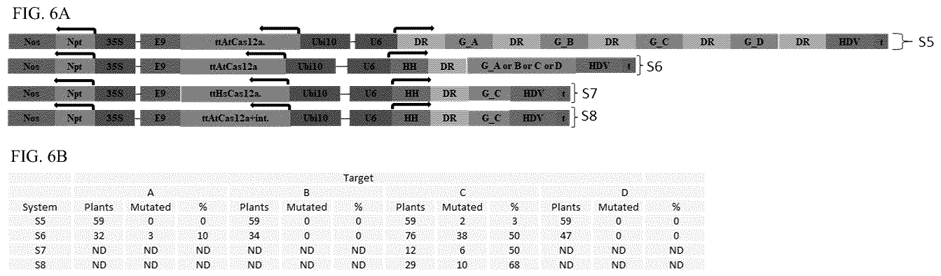

Constructs as shown in FIG. 6A were created (referred to as S5, S6, S7, and S8, herein). Briefly, S5 incorporates a guide architecture analogous to the V1 array, wherein the 4 guide RNAs are driven by one AtU626 promoter and processing of the single transcript is carried out by the Cas12a nuclease itself. S6 has an identical LbCas12a expression cassette as S5 (ttAtCas12a) but comprises a guide architecture analogous to the V2 array, wherein expression of a single guide is driven by a AtU626 promoter. As such, four S6 constructs, each containing a distinct guide RNA (A, B, C, or D) were made. The V2 guide architecture was retained in S7 using guide C in conjunction with ttHsCas12a. Similarly, S8 contained the V2 architecture using guide C, but contained the ttAtCas12+int variant. The constructs were individually transformed into B. oleracea using Agrobacterium mediated transformation and T0 plants were regenerated.

FIG. 6B shows the percent of T0 plants mutated as each target locus. From the S9 S5 T0 plants screened, just two (3%) carried targeted mutations, both of which were located at the guide C target. T0 plants transformed with S6, comprising the identical LbCas12a expression cassette with the V2 guide architecture, resulted in 10% of plants being successfully mutagenized at locus A and 50% at locus C. Thus, by changing the guide architecture alone from V1 to V2 the editing efficiency of targeted mutagenesis was increased from 0% to 10% at locus A and from 3% to 50% at locus C.

T0 plants transformed with S7 resulted in 50% of plants carrying mutations at locus C indicating that ttHsCas12a and ttAtCas12a appear to be equally efficient in B. oleracea. Additionally, the efficiency of targeted mutagenesis increased to 68% at locus C when T0 plants were transformed with S8. These results indicate that the inclusion of 8 introns into ttAtCas12a alone surprisingly increased the efficiency of targeted mutagenesis from 50% to 68%.

### Example 5. Inheritance of Edits in B. oleracea Progeny Plants

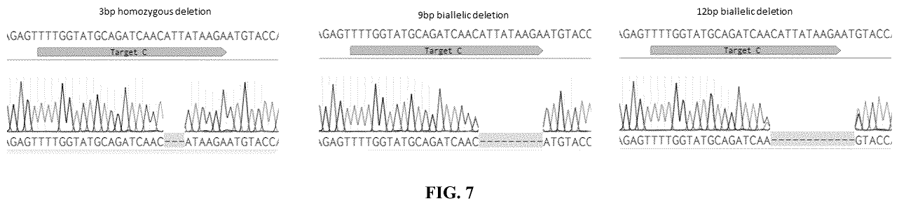

In order to ensure that LbCas12a derived mutations in B. oleracea could be passed to the next generation in the absence of T-DNA, two T0 lines with mutations at locus C were analyzed in the T1 generation. 24 seeds were germinated for each of the two T0 lines and T-DNA free progeny were identified using PCR for the Nptll marker. From the first line, 9/24 progeny did not contain the T-DNA and all were homozygous for a 3 bp deletion at locus C. From the second line progeny were T-DNA free, three of which contained 9 bp biallelic deletions and two with 12 bp biallelic deletions (FIG. 7). These results confirm that ttHsCas12a yielded the expected knockout phenotype in the first generation. These results also demonstrate that mutations resulting from LbCas12a editing in B. oleracea T0 plants show inheritance in progeny plants.

### Example 6. Evaluation of Novel Cas12a Variant Editing in Wheat Plants

Editing efficiency experiments analogous to those described in Examples 1-4 were carried out in wheat. Currently editing efficiency in wheat is believed to be very low (around 5%) with only one incidence of a substantial increase to 24%. Based on the results disclosed herein, it was expected that the ttHsCas12a and ttAtCas12a+int variants can significantly increase the efficiency of Cas12a mutagenesis in wheat to a similar level as seen in barley.

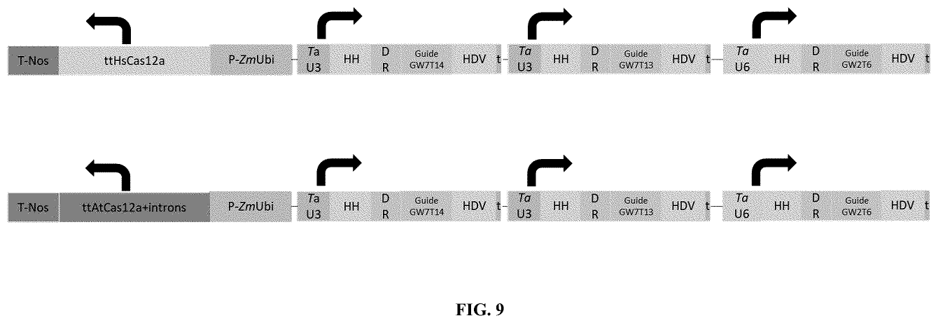

Two high-performing versions of LbCas12a, identified in the previous examples, were evaluated in wheat. Guide sequences (Wang, 2021) had been used to target various genes in conjunction with human codon optimized LbCas12a (HsCas12a) which were tested in barley as described in the previous examples. From these results, guides were identified which had resulted in mutagenesis of target genes that could be used for the present experiments. Two guides were used to target TaGW7 and one guide to target TaGW2 simultaneously using the construct architecture shown in FIG. 9.

Two constructs were made, both targeting GW7 and GW2, differing only in the LbCas12a version being used. Construct 1 contained ttHsCas12a (SEQ ID NO: 5) and construct 2 contained ttAtCas12a+8introns (SEQ ID NO: 8). Forty-eight independent wheat lines were created for each construct which were assessed by PCR and Sanger sequencing for the presence of targeted mutations in each of the three sub-genomes (A, B & D) for both GW7 and GW2 targets.

Both constructs resulted in mutagenesis in wheat and overall, as in barley, construct 2 (ttAtCas12a+8introns) was more efficient than construct 1 (ttHsCas12a). At locus GW2, 50% of ttHsCas12a lines were mutated in at least one of the 3 sub-genomes compared to 83% of ttAtCas12a+8intron lines. At the GW7 locus this figure was 75% and 94% respectively. For ttHsCas12a lines 21% were mutated in all 3 sub-genomes at the GW2 locus compared to 38% for ttAtCas12a+8introns lines. At the GW7 locus this figure was 38% and 71% respectively. Nineteen percent of ttHsCas12a lines were mutated in all 3 sub-genomes of both GW2 and GW7 loci and this figure increased to 33% in ttAtCas12a+8introns lines. Out of the 288 alleles available at both GW2 plus GW7 loci in the 48 lines created for both constructs, 44% were mutated in ttHsCas12a lines and 74% in ttAtCas12a+8introns lines.

These results indicate that ttAtCas12a+8introns performs more efficiently than ttHsCas12a in wheat.

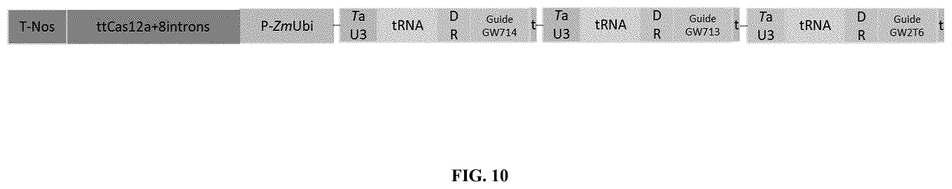

An alternate more efficient guide architecture incorporating tRNA sequences instead of ribozymes was also tested in wheat. A third construct using the ttAtCas12a+8introns nuclease with the three guide RNAs in this alternative architecture was created as shown in FIG. 10.

This architecture further improved the results, with 96% of lines containing mutations in at least one of the GW2 sub genomes and 94% of lines containing mutations in at least one of the GW7 sub genomes. Ninety percent of all 3 GW2 and 77% of all GW7 sub genomes were edited in the same lines. Seventy-three percent of lines contained mutations in all 3 sub genomes of both GW2 and GW7. Out of 288 alleles available at both GW2 and GW7 loci, 258 (90%) were edited, breaking down to 93% of GW2 alleles and 86% of GW7 alleles. In essence the biggest improvement from using the tRNA guide architecture came to the GW2 locus, possibly by making more of the GW2T6 guide transcript available in a form readily available to complex with the Cas12a nuclease.

The high efficiencies for the constructs disclosed herein were very surprising relative to previous studies conducted in protoplasts (Wang, 2001), which reported maximum efficiencies of around 14%. Previously reported stable transgenic lines included just 2/51 (4%) lines containing mutations in one sub-genome at the GW7 locus while none were reported at GW2.

In summary, the ttAtCas12a+introns construct disclosed herein has proven to be very efficient in wheat. Where two tRNA guides were used to target GW7, 86% of available alleles were mutated. Where one tRNA guide was used to target GW2, 93% of available alleles were mutated.

### Example 7. Evaluation of Novel Cas12a Variant Editing in Maize Plants

Editing efficiency experiments analogous to those described in Examples 1-4 will be carried out in corn. Currently editing efficiency in corn using LbCas12a is believed to be very low. Based on the results disclosed herein, it is expected that the ttHsCas12a and ttAtCas12a+int variants can significantly increase the efficiency of Cas12a mutagenesis in corn to a similar level as seen in barley and B. oleracea.

### Example 8. Comparison of Editing Efficiency of ttAtCas12a with and without Introns in Arabidopsis Thaliana

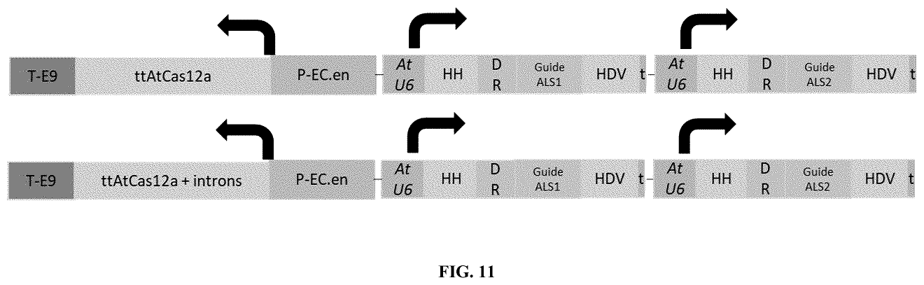

Here the efficiency of ttAtCas12a with and without introns were compared by targeting the acetolactase synthase (ALS) gene in Arabidopsis (At3g48560) using two guide RNAs in construct architecture shown in FIG. 11, where the Cas12a nuclease is driven by an egg cell specific promoter (EC.en). Egg cell expression is expected to be absent in the first-generation plants (T1) until after meiosis, where it may occur in egg cells which have segregated to contain the transgene.

Only two transgenic lines for the Cas12a version containing introns were obtained. However, this gene is likely to be lethal if knocked out completely due to its role in essential amino acid synthesis, which may cause inadvertent selection for lines where editing was less efficient.

For the two intron-containing lines (prefix 3312), 48 plants per line were screened, with 21% and 12.5% being edited at guide 1 (av.16.7%) and 67% and 52% being edited at guide 2 (av.59.5%).

Several lines were obtained for the Cas12a version which did not contain introns. For the non-intron lines (prefix 3310) sufficient seed was germinated to screen 24 T2 plants per line for 9 randomly selected lines. Efficiency varied between 0% and 17% for guide 1 and between 4% and 58% for guide 2, with an overall average efficiency of 5.1% for guide 1 and 30% for guide 2.

These results appear to indicate a better performance from the intron containing Cas12a version for the two lines evaluated. Further, the data confirmed that the version of ttCas12a with 8 introns disclosed herein functions in Arabidopsis.

### Example 9. Evaluation of Further Cas12a Variants in Barley

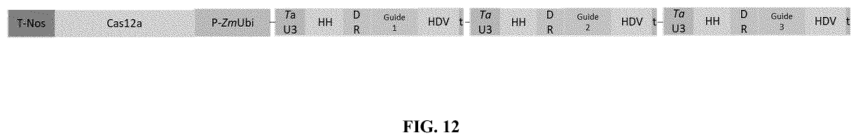

Additional constructs are assembled to further test Cas12a variants in barley. Exemplary variants have the construct architecture shown in FIG. 12. Twelve LbCas12a coding sequence (CDS) variants using the construct architecture in FIG. 12 are tested, with each construct targeting the same 3 genes, each with just one guide shown to be functional in the preceding Examples.

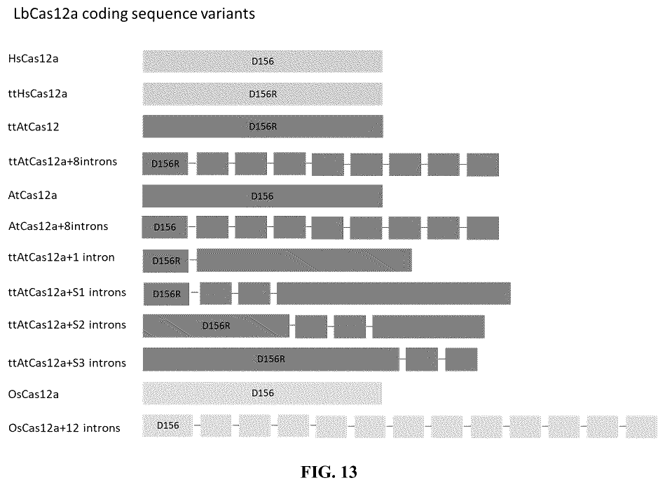

Guide 1 targets HORVU.MOREX.r3.2HG0133680, Guide 2 targets HORVU.MOREX.r3.7HG0640970, and Guide 3 targets HORVU.MOREX.r3.6HG0611290. The only difference between constructs is the coding sequence it contains. The 12 CDS's are shown in FIG. 13. Twenty independent transgenic barley plants are made for each of the 12 constructs, and these are sampled once they are large enough and screened for editing at target loci by PCR and amplicon sequencing. The efficiency of editing for the 12 CDS's over three different gene targets is determined. The editing efficiency of HsCas12a with and without D156R in barley is measured. The editing efficiency of AtCas12a with and without introns in barley is determined.

The effect on editing efficiency of HsCas12a, ttHsCas12a, and ttAtCas12a+8 introns in barley is observed for three further gene targets. Further, the effect of varying numbers of introns within Cas12a variants is determined, including comparison of AtCas12a with D156R (ttAtCas12a; SEQ ID NO:5) and ttAtCas12a+8 introns compared with ttAtCas12a+1 intron. Editing efficiency of ttAtCas12a+8 introns, ttAtCas12a+S1 introns (retaining introns 1/2/3), ttAtCas12a+S2 introns (retaining introns 4/5/6), and ttAtCas12a+S3 introns (retaining introns 7/8) is also evaluated.

A rice codon optimized Cas12a CDS (OsCas12a+12 introns; SEQ ID NO:58) is developed using various short Arabidopsis introns and gene editing efficiency of this coding sequence is evaluated in comparison with the rice-optimized Cas12a coding sequence (CDS) (OsCas12a; SEQ ID NO:1).

### Example 10. Evaluation of Further Cas12a Variants in Mammalian Cells

Three Cas12a variants, L0-Cas12a-HsD156R (human codon optimized), Picsl90022 (Arabidopsis codon optimized), and EC00968 (modified Arabidopsis codon), targeting DNMT-1, EXM1, and FANCF genes are provided as glycerol stocks in bacteria. Mammalian cells (FreeStyle™ 293-F cells, QIB Extra, Ltd.) are transfected. Expression of Cas12a is determined by dot-blot and the efficiency of the reaction assessed by flow cytometry and sequencing.

Recombinant bacterial cells carrying the plasmids with Cas12a are grown and purified. The new Cas12a recombinant plasmids are produced by cloning each of the three Cas12a inserts into the pcDNA3.1-U6 vector separately. For the crRNA plasmids, DNMT1 gRNA (SEQ ID NO: 47), EMX1 gRNA (SEQ ID NO: 48) and FANCF gRNA (SEQ ID NO: 49) are synthesized and individually cloned into pcDNA3.1-U6. In total, 6 recombinant plasmids based on pcDNA3.1-U6 vector are generated.

In order to obtain sufficient purified recombinant plasmids for mammalian cell transfection the recombinant plasmids generated above are transformed into competent NEB® 10-beta competent E. coli cells using the heat shock protocol. Super optimal broth with catabolite suppression is added to the cells and incubated at 37° C. The suspension is spread on LB plates containing carbenicillin. Colonies for each transformation reaction are selected and grown in LB broth and the recombinant plasmids will be purified using the PureLink™ HiPure Plasmid Miniprep Kit and a sample is analyzed on agarose gel electrophoresis following restriction digest to verify the integrity of the recombinant plasmids.

FreeStyle™ 293-F cells are seeded in a 48-well plate with antibiotic-free medium 16 h prior to transfection (1 plate per construct). Cells are co-transfected with each recombinant Cas12a plasmid together with each crRNA recombinant plasmid using Lipofectamine 2000, resulting in 9 types of co-transfections. Cells transfected with the relevant Cas12a plasmid only are used as negative control. To test transfection efficiency and Cas12a expression, co-transfection of the three Cas12a plasmids with the DNMT1 gRNA target is performed. Control transfections are performed with the Cas12a plasmids only. Following an 8 h incubation, the transfection medium is removed and replaced with fresh medium. Following 72 h incubation, cells are checked for Cas12a expression by antibody detection. Briefly, transfected or control cells are lysed and the extracted proteins are analyzed by dot blot using first a mouse anti-lbCas12a antibody and an anti-mouse IgG-HRP conjugated secondary antibody. Depending on results, the transfection conditions are optimized before moving to the other co-transfection combinations.

To analyze target gene cleavage, sequencing is used to monitor EMX1 and FANCF cleavage while DNMT1 cleavage is determined by both sequencing and flow cytometry (due to the availability of a suitable commercial antibody for this target). For the flow cytometry, transfected cells expressing Cas12a (generated from Step 3) are first be stained with a viability dye (Zombie Fixable Viability), then fixed and permeabilized using a Fixation/Permeabilization Buffer and finally, cells are incubated with an anti-DNMT1-PE antibody. For the sequencing approach, FreeStyle™ 293-F cell genomic DNA is purified and used as a template for PCR using specific primers against a gene region of the target site. The PCR product will be further purified using a DNA extraction kit (Qiagen Gel extraction kit, Qiagen) and sequenced at an in-house sequencing facility.

What is claimed is:

1. A recombinant DNA molecule comprising a polynucleotide sequence selected from the group consisting of:
a. a sequence with at least 85 percent identity to any of SEQ ID NOs:1, 3, 5, 7, and 8;
b. a sequence comprising SEQ ID NOs:1, 3, 5, 7, and 8;
c. a fragment of a sequence having at least 85 percent sequence identity to any of SEQ ID NOs:1, 3, 5, 7, and 8, wherein the fragment has nuclease activity;
c. a fragment of any of SEQ ID NOs:1, 3, 5, 7, and 8; and
d. a sequence encoding a protein having at least 85 percent identity to any of SEQ ID NOs: 2, 4, 6, and 9;
wherein the protein encoded by said polynucleotide sequence comprises a modification at amino acid position 156 as compared to a protein comprising the amino acid sequence of SEQ ID NO:46 and at least one intron sequence having a sequence having at least 85 percent identity to any one of SEQ ID NOs:10-17 or functional fragment thereof.

2. The recombinant DNA molecule of claim 1, wherein said sequence has at least 90 percent identity to any of SEQ ID NOs:1, 3, 5, 7, and 8 and encodes a protein having a modification at amino acid position 156 as compared to a protein comprising the amino acid sequence of SEQ ID NO:46.

3. The recombinant DNA molecule of claim 2, wherein said sequence has at least 95 percent identity to any of SEQ ID NOs:1, 3, 5, 7, and 8 and encodes a protein having a modification at amino acid position 156 as compared to a protein comprising the amino acid sequence of SEQ ID NO:46.

4. The recombinant DNA molecule of claim 1, wherein said sequence comprises any of SEQ ID NOs:1, 3, 5, 7, and 8.

5. The recombinant DNA molecule of claim 1, wherein the modification at amino acid position 156 is further defined as an aspartate to arginine substitution.

6. The recombinant DNA molecule of claim 1, wherein said polynucleotide sequence further comprises intron sequences of SEQ ID NOs:10-17.

7. A transgenic plant cell comprising the recombinant DNA molecule of claim 1.

8. The transgenic plant cell of claim 7, wherein said transgenic plant cell is a monocotyledonous plant cell.

9. The transgenic plant cell of claim 8, wherein said monocotyledonous plant cell is selected from the group consisting of a barley, B. oleracea, wheat, and corn cell.

10. The transgenic plant cell of claim 7, wherein said transgenic plant cell is a dicotyledonous plant cell.

11. A transgenic plant, or part thereof, comprising the recombinant DNA molecule of claim 1.

12. A progeny plant of the transgenic plant of claim 11, or a part thereof, wherein the progeny plant or part thereof comprises said recombinant DNA molecule.

13. A transgenic seed, wherein the seed comprises the recombinant DNA molecule of claim 1.

14. The recombinant DNA molecule of claim 1, wherein:
a. said recombinant DNA molecule is expressed in a plant cell to produce a genomic modification; or
b. said recombinant DNA molecule is in operable linkage with a vector, and said vector is selected from the group consisting of a plasmid, phagemid, bacmid, cosmid, and a bacterial or yeast artificial chromosome.

15. The recombinant DNA molecule of claim 14, present within a host cell, wherein said host cell is selected from the group consisting of a bacterial cell and a plant cell.

16. The recombinant DNA molecule of claim 15, wherein said bacterial host cell is from a genus of bacteria selected from the group consisting of: Agrobacterium, Rhizobium, Bacillus, Brevibacillus, Escherichia, Pseudomonas, Klebsiella, Pantoea, and Erwinia.

17. The recombinant DNA of claim 15, wherein said plant cell is a dicotyledonous or a monocotyledonous plant cell.

18. The recombinant DNA of claim 17, wherein said plant cell is selected from the group consisting of a Fabaceae, sunflower, safflower, sesame, tobacco, potato, cotton, sweet potato, cassava, coffee, tea, apple, pear, fig, citrus tree, cocoa, avocado, olive, almond, walnut, strawberry, watermelon, pepper, beet, grape, tomato, cucumber, thale cress, Brassica sp., pea, alfalfa, barrel clover, pigeon pea, guar, carob, fenugreek, soybean, common bean, cowpea, mung bean, lima bean, fava bean, lentil, peanut, licorice, chickpea, oil palm, coconut, banana, corn, barley, sorghum, rice, and wheat cell.

19. A method for producing a plant comprising a genomic modification, the method comprising:
a. expressing the recombinant DNA molecule of claim 1 and a guide RNA compatible with the protein encoded by said recombinant DNA molecule in a plant cell;
b. introducing a modification into at least one target site in the plant cell genome;
c. identifying and selecting one or more plant cells of step (b) comprising said modification in said plant genome; and
d. regenerating at least one plant from at least one or more cells selected in step (c).

20. The method of claim 19, wherein the modification is selected from the group consisting of a substitution, an insertion, an inversion, a deletion, a duplication, and a combination thereof.

21. The method of claim 19, wherein the plant is a monocotyledonous plant.

22. The method of claim 21, wherein the plant is selected from the group consisting of a barley, B. oleracea, wheat, and corn plant.

23. A method of producing progeny seed comprising the recombinant DNA molecule of claim 1, the method comprising:
a. planting a first seed comprising the recombinant DNA molecule of claim 1;
b. growing a plant from the seed of step (a); and
c. harvesting the progeny seed from the plants, wherein said harvested seed comprises said recombinant DNA molecule.

24. A method for introducing a genomic modification in a plant, said method comprising:
a. expressing a protein or fragment thereof encoded by the DNA molecule of claim 1 in a plant; and
b. expressing a guide RNA compatible with said protein or fragment thereof having nuclease activity in a plant cell.

25. A method of detecting the presence of the recombinant DNA molecule of claim 1 in a sample comprising plant genomic DNA, comprising:
a. contacting said sample with a DNA probe that hybridizes under stringent hybridization conditions with genomic DNA from a plant comprising the recombinant nucleic DNA of claim 1, and does not hybridize under such hybridization conditions with genomic DNA from an otherwise isogenic plant that does not comprise the recombinant DNA molecule of claim 1, wherein said probe is homologous or complementary to a fragment of any of SEQ ID NOs:1, 3, 5, 7, 8; or a sequence that encodes a protein comprising an amino acid sequence having at least 85%, or 90%, or 95%, or 98% or 99%, or about 100% amino acid sequence identity to any of SEQ ID NOs: 2, 4, 6, and 9;
b. subjecting said sample and said probe to stringent hybridization conditions; and
c. detecting hybridization of said DNA probe with said recombinant DNA molecule.

26. A method of detecting the presence of a nuclease protein, or fragment thereof, in a sample comprising protein, wherein said protein comprises the amino acid sequence of any of SEQ ID NOs: 2, 4, 6, and 9 or fragment thereof; or said protein comprises an amino acid sequence having at least 85%, or 90%, or 95%, or 98% or 99%, or about 100% amino acid sequence identity to any of SEQ ID NOs: 2, 4, 6, and 9 or fragment thereof; comprising:
a. contacting said sample with an immunoreactive antibody; and
b. detecting the presence of said protein, or fragment thereof.

27. A method for modifying a polynucleotide segment encoding a Cas12a protein or fragment thereof having nuclease activity, the method comprising:
a. obtaining a polynucleotide sequence of any of SEQ ID NOs:1, 3, 5, 7 and 8; and
b. introducing a modification into at least one target site in the polynucleotide sequence such that the protein encoded by said polynucleotide sequence comprises a modification at amino acid position 156 as compared to a protein comprising the amino acid sequence of SEQ ID NO: 46;
wherein the modified polynucleotide sequence further comprises at least one intron sequence having a sequence having at least 85 percent identity to any one of SEQ ID NOs:10-17 or functional fragment thereof.

28. The method of claim 27, wherein the protein encoded by the modified polynucleotide sequence comprises an aspartate to arginine substitution at amino acid position 156 as compared to a polynucleotide segment lacking said modification.

29. The method of claim 28, wherein the modified polynucleotide sequence further comprises intron sequences of SEQ ID NO:10-17.

30. The method of claim 27, wherein the modified polynucleotide sequence comprises an aspartate to arginine modification at amino acid position 156 and further comprises at least one intron sequence of SEQ ID NOs:10-17.

31. A method for improving gene targeting using CRISPR-Cas12a gene editing in crops, comprising the steps of:
a. expressing the recombinant DNA molecule of claim 1 and a guide RNA compatible with the protein encoded by said recombinant DNA molecule in a plant cell; and
b. introducing a modification into at least one target site in the plant cell genome;
wherein said modification is introduced at a higher rate when compared to the rate of introduction of a modification using a method comprising expressing a DNA molecule encoding the amino acid of SEQ ID NO:46.

32. The method of claim 31, wherein the sequence has at least 90 percent identity to any of SEQ ID NOs:1, 3, 5, 7, and 8 and encodes a protein having a modification at amino acid position 156 as compared to a protein comprising the amino acid sequence of SEQ ID NO:46.

33. The method of claim 32, wherein the sequence has at least 95 percent identity to any of SEQ ID NOs:1, 3, 5, 7, and 8 and encodes a protein having a modification at amino acid position 156 as compared to a protein comprising the amino acid sequence of SEQ ID NO:46.

34. The method of claim 31, wherein the sequence comprises any of SEQ ID NOs:1, 3, 5, 7, and 8.

35. The method of claim 31, wherein the modification at amino acid position 156 is further defined as an aspartate to arginine substitution.

36. The method of claim 31, wherein the polynucleotide sequence further comprises intron sequences of SEQ ID NOs:10-17.


SEQUENCE LISTING (58 sequences)

SEQ ID NO: 1 - DNA, 3684 bp, synthetic construct
  source 1..3684: mol_type = other DNA; note = Codon-optimized sequence
gcggcttcaaagctcgagaaattcaccaactgttattcgttgagcaaaacactgcggtttaaagcgattccagtcggcaagactcaagagaatatagacaataagcggctgttggtggaagatgaaaagcgcgcggaggactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaaaacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaggacgaaaaattggacaaggtcaaaatcgctatcagcaacaaagagtggctggagtatgctcagacatccgtaaag

SEQ ID NO: 2 - protein, 1228 aa, synthetic construct
  source 1..1228: note = Recombinant amino acid sequence
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 3 - DNA, 3684 bp, synthetic construct
  source 1..3684: mol_type = other DNA; note = Codon-optimized sequence
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 4 - protein, 1227 aa, synthetic construct
  source 1..1227: note = Recombinant amino acid sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 5 - DNA, 3684 bp, synthetic construct
  source 1..3684: mol_type = other DNA; note = Codon-optimized sequence
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttcgcaacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 6 - protein, 1227 aa, synthetic construct
  source 1..1227: note = Recombinant amino acid sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 7 - DNA, 3684 bp, synthetic construct
  source 1..3684: mol_type = other DNA; note = Codon-optimized sequence
atgagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcagaaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaagcttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctggacaaggtgaagatcgccatctccaacaaagagtggcttgagtacgctcagacctccgttaagcac

SEQ ID NO: 8 - DNA, 4622 bp, synthetic construct
  source 1..4622: mol_type = other DNA; note = Codon-optimized sequence
atgagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcagaaacagggaaaacatgttcagcgaagaggccaagagcacctctatcgctttcagatgcatcaacgagaacctcacgcgttacatcagcaacatggacatcttcgagaaggtaacattccttagttacctttcttttctttttccatcataagtttatagattgtacatgctttgagatttttctttgcaaacaatctcaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggtctgtctttcctatttcatatgtttaatcctaggaatttgatcaattgattgtatgtatgtcgatcccaagactttcttgttcacttatatcttaactctctctttgctgtttcttgcaggttctctctgatcgtgagagcctctcattttacggtgagggttacacctctgacgaggaagtgcttgaggttttccgtaacaccctcaacaagaacagcgagatcttctcgtccatcaagaagttggagaagcttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtaagttgttacttatgattgttttcctctctgctacatgtattttgttgttcatttctgtaagatataagaattgagttttcctctgatgatattattaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggttagtatcatatgaagaaatacctagtttcagttgatgaatgctattttctgacctcagttgttctcttttgagaattatttcttttctaatttgcctgatttttctattaattcattaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaagttagcttcgagagcgcctccaagaaagaggtaaatcctggtccacacttttacgataaaaacacaagattttaaactatgaactgatcaataatcattcctaaaagaccacacttttgttttgtttctaaagtaatttttactgttataacaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtaaggacttctcatgaatattagtggcagattagtgttgttaaagtctttggttagataatcgatgcctcctaattgtccatgttttactggttttctacaattaaaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggtaagttctgcatttggttatgctccttgcattttaggtgttcgtcgcacttccatttccatgaatagctaagattttttttctctgcattcattcttcttgcctcagttctaactgtttgtggtatttttgttttaattattgctacaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggtaaagcaactgtgttttaatcaatttcttgtcaggatatatggattataacttaatttttgagaaatctgtagtatttggcgtgaaatgagtttgctttttggtttctcccgtgttataggttctctgggctatcgggcagtttaagaaagcggaagatgagaagctggacaaggtgaagatcgccatctccaacaaagagtggcttgagtacgctcagacctccgttaagcac

SEQ ID NO: 9 - protein, 1227 aa, synthetic construct; identical to SEQ ID NO: 6
  source 1..1227: note = Recombinant amino acid sequence

SEQ ID NO: 10 - DNA, 91 bp, Arabidopsis thaliana
  source 1..91: mol_type = unassigned DNA
gtaacattccttagttacctttcttttctttttccatcataagtttatagattgtacatgctttgagatttttctttgcaaacaatctcag

SEQ ID NO: 11 - DNA, 121 bp, Arabidopsis thaliana
  source 1..121: mol_type = unassigned DNA
gtctgtctttcctatttcatatgtttaatcctaggaatttgatcaattgattgtatgtatgtcgatcccaagactttcttgttcacttatatcttaactctctctttgctgtttcttgcag

SEQ ID NO: 12 - DNA, 101 bp, Arabidopsis thaliana
  source 1..101: mol_type = unassigned DNA
gtaagttgttacttatgattgttttcctctctgctacatgtattttgttgttcatttctgtaagatataagaattgagttttcctctgatgatattattag

SEQ ID NO: 13 - DNA, 121 bp, Arabidopsis thaliana
  source 1..121: mol_type = unassigned DNA
gttagtatcatatgaagaaatacctagtttcagttgatgaatgctattttctgacctcagttgttctcttttgagaattatttcttttctaatttgcctgatttttctattaattcattag

SEQ ID NO: 14 - DNA, 124 bp, Arabidopsis thaliana
  source 1..124: mol_type = unassigned DNA
gtaaatcctggtccacacttttacgataaaaacacaagattttaaactatgaactgatcaataatcattcctaaaagaccacacttttgttttgtttctaaagtaatttttactgttataacag

SEQ ID NO: 15 - DNA, 109 bp, Arabidopsis thaliana
  source 1..109: mol_type = unassigned DNA
gtaaggacttctcatgaatattagtggcagattagtgttgttaaagtctttggttagataatcgatgcctcctaattgtccatgttttactggttttctacaattaaag

SEQ ID NO: 16 - DNA, 150 bp, Arabidopsis thaliana
  source 1..150: mol_type = unassigned DNA
gtaagttctgcatttggttatgctccttgcattttaggtgttcgtcgcacttccatttccatgaatagctaagattttttttctctgcattcattcttcttgcctcagttctaactgtttgtggtatttttgttttaattattgctacag

SEQ ID NO: 17 - DNA, 121 bp, Arabidopsis thaliana
  source 1..121: mol_type = unassigned DNA
gtaaagcaactgtgttttaatcaatttcttgtcaggatatatggattataacttaatttttgagaaatctgtagtatttggcgtgaaatgagtttgctttttggtttctcccgtgttatag

SEQ ID NO: 18 - DNA, 643 bp, synthetic construct
  source 1..643: mol_type = other DNA; note = Guide RNA array
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgtaatttctactaagtgtagatnnnnnnnnnnnnnnnnnnnnnnntaatttctactaagtgtagatnnnnnnnnnnnnnnnnnnnnnnntaatttctactaagtgtagatnnnnnnnnnnnnnnnnnnnnnnntaatttctactaagtgtagatnnnnnnnnnnnnnnnnnnnnnnntaatttctactaagtgtagatgtcccttcgaagggcaattctgcagatatccatcacactggcggccgctcgaggtcgagggtatcgataagcttttttttttt

SEQ ID NO: 19 - DNA, 2172 bp, synthetic construct
  source 1..2172: mol_type = other DNA; note = Guide RNA array
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgaaattactgatgagtccgtgaggacgaaacgagtaagctcgtctaatttctactaagtgtagatnnnnnnnnnnnnnnnnnnnnnnnggccggcatggtcccagcctcctcgctggcgccggctgggcaacatgcttcggcatggcgaatgggactttttgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgaaattactgatgagtccgtgaggacgaaacgagtaagctcgtctaatttctactaagtgtagatnnnnnnnnnnnnnnnnnnnnnnnggccggcatggtcccagcctcctcgctggcgccggctgggcaacatgcttcggcatggcgaatgggactttttttacggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagccctaccaacctcacgggccagcaattacgagtccttctaaaacgtcccgccgagggcgcgtggccgtgctgtgcagcagcacgtctaacattagtcccacctcgccagtttacagggagcagaaccagcttataagcggaggcgcggcaccaagaagcaaaattactgatgagtccgtgaggacgaaacgagtaagctcgtctaatttctactaagtgtagatnnnnnnnnnnnnnnnnnnnnnnnggccggcatggtcccagcctcctcgctggcgccggctgggcaacatgcttcggcatggcgaatgggactttttggcggcagggagagttttaacattgactagcgtgctgataatttgtgagaaataataattgacaagtagatactgacatttgagaagagcttctgaactgttattagtaacaaaaatggaaagctgatgcacggaaaaaggaaagaaaaagccatacttttttttaggtaggaaaagaaaaagccatacgagactgatgtctctcagatgggccgggatctgtctatctagcaggcagcagccctaccaacctcacgggccagcaattacgagtccttctaaaacgtcccgccgagggcgcgtggccgtgctgtgcagcagcacgtctaacattagtcccacctcgccagtttacagggagcagaaccagcttataagcggaggcgcggcaccaagaagcaaaattactgatgagtccgtgaggacgaaacgagtaagctcgtctaatttctactaagtgtagatnnnnnnnnnnnnnnnnnnnnnnnggccggcatggtcccagcctcctcgctggcgccggctgggcaacatgcttcggcatggcgaatgggacttttt

SEQ ID NO: 20 - DNA, 23 bp, synthetic construct
  source 1..23: mol_type = other DNA; note = Guide RNA
tccatagtgagaagaggtgtgag

SEQ ID NO: 21 - DNA, 23 bp, synthetic construct
  source 1..23: mol_type = other DNA; note = Guide RNA
tcgcgacctgggtctttccttca

SEQ ID NO: 22 - DNA, 23 bp, synthetic construct
  source 1..23: mol_type = other DNA; note = Guide RNA
gtgctgcacaatgtcaacaactg

SEQ ID NO: 23 - DNA, 23 bp, synthetic construct
  source 1..23: mol_type = other DNA; note = Guide RNA
acccctgctgcatccccatcatc

SEQ ID NO: 24 - DNA, 23 bp, synthetic construct
  source 1..23: mol_type = other DNA; note = Guide RNA
tggaggatggcggcgtcgtgggc

SEQ ID NO: 25 - DNA, 23 bp, synthetic construct
  source 1..23: mol_type = other DNA; note = Guide RNA
tcagtaagcatgttcaagaacgg

SEQ ID NO: 26 - DNA, 23 bp, synthetic construct
  source 1..23: mol_type = other DNA; note = Guide RNA
agcggggcagacgtgctggggcg

SEQ ID NO: 27 - DNA, 23 bp, synthetic construct
  source 1..23: mol_type = other DNA; note = Guide RNA
atgctaaaaaacactcgaccacg

SEQ ID NO: 28 - DNA, 23 bp, synthetic construct
  source 1..23: mol_type = other DNA; note = Guide RNA
tcacacgaactgccggaacgttg

SEQ ID NO: 29 - DNA, 23 bp, synthetic construct
  source 1..23: mol_type = other DNA; note = Guide RNA
aaggacatgatcatggagggtga

SEQ ID NO: 30 - DNA, 23 bp, synthetic construct
  source 1..23: mol_type = other DNA; note = Guide RNA
gacctacaagacctacaagtgtg

SEQ ID NO: 31 - DNA, 23 bp, synthetic construct
  source 1..23: mol_type = other DNA; note = Guide RNA
attcatataagcttgtactactt

SEQ ID NO: 32 - DNA, 23 bp, synthetic construct
  source 1..23: mol_type = other DNA; note = Guide RNA
tgaaccccatcatctccatgctc

SEQ ID NO: 33 - DNA, 23 bp, synthetic construct
  source 1..23: mol_type = other DNA; note = Guide RNA
tgttcttccttgaccagcatcat

SEQ ID NO: 34 - DNA, 23 bp, synthetic construct
  source 1..23: mol_type = other DNA; note = Guide RNA
ggaactatttcagtcaatcatcg

SEQ ID NO: 35 - DNA, 23 bp, synthetic construct
  source 1..23: mol_type = other DNA; note = Guide RNA
ctcgaaaggaggaagtgtcaaga

SEQ ID NO: 36 - DNA, 23 bp, synthetic construct
  source 1..23: mol_type = other DNA; note = Guide RNA
ccatagctcctccttgagtcgct

SEQ ID NO: 37 - DNA, 23 bp, synthetic construct
  source 1..23: mol_type = other DNA; note = Guide RNA
aggctgagagatggtgcgccatt

SEQ ID NO: 38 - DNA, 23 bp, synthetic construct
  source 1..23: mol_type = other DNA; note = Guide RNA
ttctgacctgatgtggagttctg

SEQ ID NO: 39 - DNA, 23 bp, synthetic construct
  source 1..23: mol_type = other DNA; note = Guide RNA
gcagtgaagaggaaaattaaacg

SEQ ID NO: 40 - DNA, 45 bp, synthetic construct
  source 1..45: mol_type = other DNA; note = Recombinant nuclear localization sequence
atggctcctaagaagaagcggaaggttggtattcacggggtccct

SEQ ID NO: 41 - protein, 15 aa, synthetic construct
  source 1..15: note = Recombinant nuclear localization sequence
MAPKKKRKVGIHGVP

SEQ ID NO: 42 - DNA, 51 bp, synthetic construct
  source 1..51: mol_type = other DNA; note = Recombinant nuclear localization sequence
cataagcgtcctgctgccaccaaaaaggccggacaggctaagaaaaagaag

SEQ ID NO: 43 - protein, 17 aa, synthetic construct
  source 1..17: note = Recombinant nuclear localization sequence
HKRPAATKKAGQAKKKK

SEQ ID NO: 44 - DNA, 48 bp, synthetic construct
  source 1..48: mol_type = other DNA; note = Recombinant nuclear localization sequence
aaaaggccggcggccacgaaaaaggccggccaggcaaaaaagaaaaag

SEQ ID NO: 45 - DNA, 48 bp, synthetic construct
  source 1..48: mol_type = other DNA; note = Recombinant nuclear localization sequence
aagaggcctgctgctactaagaaagctggtcaggctaagaagaagaaa

SEQ ID NO: 46 - protein, 1227 aa, unidentified
  source 1..1227: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 47 - RNA, 23 nt, synthetic construct
  source 1..23: mol_type = other RNA
ctgatggtccatgtctgttactc

SEQ ID NO: 48 - RNA, 23 nt, synthetic construct
  source 1..23: mol_type = other RNA
tcatctgtgcccctccctccctg

SEQ ID NO: 49 - RNA, 23 nt, synthetic construct
  source 1..23: mol_type = other RNA
accttggagacggcgactctctg

SEQ ID NO: 50 - DNA, 29 bp, synthetic construct
  source 1..29: mol_type = other DNA
tttggtgctgcacaatgaaagcagacggc

SEQ ID NO: 51 - DNA, 36 bp, synthetic construct
  source 1..36: mol_type = other DNA
tttggtgctgcacaacaacaactgaaagcagacggc

SEQ ID NO: 52 - DNA, 39 bp, Hordeum vulgare
  source 1..39: mol_type = genomic DNA
tttggtgctgcacaatgtcaacaactgaaagcagacggc

SEQ ID NO: 53 - DNA, 43 bp, synthetic construct
  source 1..43: mol_type = other DNA
gttgatggttggtgttgggcaatgcccaatgaaagcagacggc

SEQ ID NO: 54 - DNA, 38 bp, Brassica oleracea
  source 1..38: mol_type = genomic DNA
gagttttggtatgcagatcaacattataagaatgtacc

SEQ ID NO: 55 - DNA, 35 bp, synthetic construct
  source 1..35: mol_type = other DNA
gagttttggtatgcagatcaacataagaatgtacc

SEQ ID NO: 56 - DNA, 29 bp, synthetic construct
  source 1..29: mol_type = other DNA
gagttttggtatgcagatcaacatgtacc

SEQ ID NO: 57 - DNA, 26 bp, synthetic construct
  source 1..26: mol_type = other DNA
gagttttggtatgcagatcaagtacc

SEQ ID NO: 58 - DNA, 7432 bp, synthetic construct
  source 1..7432: mol_type = other DNA
tgagaccacgaagtggctcttcagtggacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagcgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccattacgtaaaaggcgagatcaccaaggtagtcggcaaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatcactctgtggtctcaaatggctcctaagaagaagcggaaggttggtattcacggggtccctgcggcttcaaagctcgagaaattcaccaactgttattcgttgagcaaaacactgaggtaacattccttagttacctttcttttctttttccatcataagtttatagattgtacatgctttgagatttttctttgcaaacaatctcaggtttaaagcgattccagtcggcaagactcaagagaatatagacaataagcggctgttggtggaagatgaaaagcgcgcggaggactacaaaggggtgaagaagttgttggacagatactacctctcttttatcaatgatgtcttgcactcaatcaaattgaagaatctgaacaactacatctccctcttccgcaagaaaacaaggacagaaaaggagaataaggaacttgaaaatttggagatcaatctgaggaaagagatcgcgaaagcctttaaaggtgatatatcttctttgctctattacatattggttgtccatttgttgtctcttttcttcattactcaagacaaaattagatctctttcgttgtgtactcattgtttggtttctccaggtaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtttgtttccctctcaaaacctgttctatgtttagccttagatgttgaggttttgtttgttgcatctctgatatgcaattgttataggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggtaaagtttccaactttcctttaccatatcaaactaaagttcgaaactttttatttgatcaacttcaaggccacccgatctttctattcctgattaatttgtgatgaatccatattgacttttgatggttacgcaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatatcttttcaacacactcatttcttaaactacaccaaagtctaaaagctcactaactcacttctttgatccacaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtaaatcctggtccacacttttacgataaaaacacaagattttaaactatgaactgatcaataatcattcctaaaagaccacacttttgttttgtttctaaagtaatttttactgttataacaggtggaagagcagggctacaaggtgtcattcgagtccgccagcaagaaggaagtggacaagctcgtggaagagggcaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtaagaggtcaaaaggtttccgcaatgatccctctttttttgtttctctagtttcaagaatttgggtatatgactaacttctgagtgttccttgatgcatatttgtgatgagacaaatgtttgttctatgttttaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtaagttctgcatttggttatgctccttgcattttaggtgttcgtcgcacttccatttccatgaatagctaagattttttttctctgcattcattcttcttgcctcagttctaactgtttgtggtatttttgttttaattattgctacaggtctatcaaaagttcgagaagatgcttatagataagctcaactacatggtcgataagaaatcgaacccatgtgccaccggcggcgcactcaaaggtaacttcttcctcctctttgcttttttttaccatttgaattcttttttgtctatgcaattctctgataaggtcttgtgtgttgatcaggttaccaaataacaaacaaattcgagtccttcaaatcgatgagtactcagaatgggttcatattttatataccggcgtggcttacgtctaagatcgacccgtcaactggttttgtcaacctgttgaaaacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggtatgtgttcaaaatctcaactttcaatacattttcatagttggttttggtactaatcccttggtttatctctatgtggtctaatgtgattaatactatgcttcaatctgcaggtagaaccgacgtggatttcttgatctccccggttaaaaattctgatggcatcttttacgatagccggaactatgaagcgcaagagaatgcgattctgccaaaaaatgcagacgccaacggtgcctataacatcgccaggaaagtcctgtgggcgatcggccagttcaaaaaggccgaggacgaaaaattggacaaggtaaagcaactgtgttttaatcaatttcttgtcaggatatatggattataacttaatttttgagaaatctgtagtatttggcgtgaaatgagtttgctttttggtttctcccgtgttataggtcaaaatcgctatcagcaacaaagagtggctggagtatgctcagacatccgtaaagcataagcgtcctgctgccaccaaaaaggccggacaggctaagaaaaagaagtgagctt